(19) United States
(12) Patent Application Publication
(10) Pub. No.: US 2008/0166303 A1
(43) Pub. Date: Jul. 10, 2008

(54) Colored or colorable foamable composition and foam

(76) Inventors: Dov Tamarkin, Maccabim (IL); Doron Friedman, Karmei Yosef (IL); Meir Eini, Ness Ziona (IL)

Correspondence Address: WILMERHALE/BOSTON, 60 STATE STREET, BOSTON, MA, 02109, US

(21) Appl. No.: 11/900,328

(22) Filed: Sep. 10, 2007

Related U.S. Application Data
(60) Provisional application No. 60/843,144, filed on Sep. 8, 2006.

(51) Int. Cl. A61K 9/12; A61P 17/00

(52) U.S. Cl. 424/43

(57) ABSTRACT

There is described a colored or colorable topical composition, comprising: a) a foamable base composition comprising, 1) a flowable carrier composition; 2) a color agent; wherein the color agent is effective to impart, increase, decrease or otherwise affect color of a foam produced from the foamable composition and wherein the color agent is one or more agents selected from the group consisting of a colored active agent, a colored indicator, a colored excipient, a pigment, a dye, a colorant and a coloring agent; b) a propellant at a concentration of about 3% to about 25% by weight of the total composition; wherein the base composition has a first color; and wherein the foam comprising the colored or colorable topical composition has a second color upon dispensing from an aerosol container, and wherein the first color and the second color are visually different. There is also described a method of changing color, a method of administration, a use as a diagnostic and a kit.

### CROSS REFERENCE TO RELATED APPLICATIONS

This application claims the benefit of priority under 35 U.S.C. §119(e) to U.S. Patent Application Ser. No. 60/843,144, filed Sep. 8, 2006, entitled “Colored or Colorable Topical Composition Foam,” which is herein incorporated by reference in its entirety.

### BACKGROUND

This invention relates to foamable pharmaceutical and cosmetic compositions, containing an active agent, having high color intensity.

Certain pharmaceutical and cosmetic active agents are colored and occasionally possess high color intensity. Examples of such agents are iodine and tetracycline. Additionally, many natural extracts also have high color intensity. The incorporation of such agents in semi-solid dosage forms for topical application, such as creams, ointments, gels and lotions results in products with high color intensity, which are not acceptable for the user. An exemplary drug that has strong yellow color is tetracycline, a broad range antibiotic that could be useful for the treatment of various skin infections, including acne. However, due to its intensive color, patients are reluctant to use semi-solid preparations containing such a drug.

Color may also be used as an indicator of change in a physical parameter like pH or in reaction to light or as a diagnostic upon a reaction with a target. Use of color in foam as an indicator is discussed.

Foams are considered a more convenient vehicle for topical delivery of active agents. There are several types of topical foams, including aqueous foams, such as commonly available shaving foams; hydroalcoholic foams, emulsion-based foams, comprising oil and water components, oleaginous foams, which consist of high oil content, and waterless foam.

The surprising effect of foam on color is explored and disclosed herein.

### SUMMARY

Colored or colorable compositions, which can be for topical application, and which when dispensed from an aerosol change color are provided as well as foamable compositions for use as colored or colorable topical compositions, methods of changing the color of colored or colorable topical compositions, a method of treating a disorder in a mammalian subject by administering said compositions to a target site, a colored or colorable composition kit, and use of such compositions as a diagnostic.

In one or more embodiments, there is provided a colored or colorable topical composition, comprising:
a. a foamable base composition comprising
   i. a flowable carrier composition;
   ii. a color agent;
      1. wherein the color agent is effective to impart, increase, decrease or otherwise affect color of a foam produced from the foamable composition, and
      2. wherein the color agent is one or more agents selected from the group consisting of a colored active agent, a colored indicator, a colored excipient, a pigment, a dye, a colorant and a coloring agent;
b. a propellant at a concentration of about 3% to about 25% by weight of the total composition,
wherein the base composition has a first color; and
wherein the foam comprising the colored or colorable topical composition has a second color upon dispensing the topical composition from an aerosol container, and
wherein the first color and the second color are visually different.

In one or more embodiments, there is provided a method of changing the color of a colored or colorable topical composition, comprising:
a. selecting a color agent, a flowable carrier composition a propellant and an aerosol canister; and
b. preparing a colored foamable base composition of a first color, comprising:
   i. a flowable carrier composition; and
   ii. a color agent;
wherein the agent is effective to impart, increase, decrease or otherwise affect color of a foam produced from the foamable composition; and
wherein the agent is one or more agents selected from the group consisting of a colored active agent, a colored excipient, a pigment, a dye, a colorant and a coloring agent;
c. filling the foamable base composition in the aerosol container, closing the container having an aerosol valve, adding a propellant at a concentration of about 3% to about 25% by weight of the total composition;
d. opening the aerosol valve to release foam of a second color,

wherein the first color and the second color are visually different.

In one or more embodiments, there is provided a method of treating a disorder of a mammalian subject to achieve an improved compliance, comprising:
administering a colored or colorable topical composition to a target site, the colored topical composition, comprising:
   a. a foamable base composition comprising
      i. a flowable carrier composition; and
      ii. a color agent
         i. wherein the color agent is effective to impart, increase, decrease or otherwise affect color of a foam produced from the foamable composition; and
         ii. wherein the color agent is one or more agents selected from the group consisting of a colored active agent, a colored indicator, a colored excipient, a pigment, a dye, a colorant and a coloring agent; and;
         iii. wherein the color agent comprises at least an effective amount of active agent;
   b. a propellant at a concentration of about 3% to about 25% by weight of the total composition;
wherein the base composition has a first color; and
wherein the colored topical composition has a second color after it has been dispensed from an aerosol container, and
wherein the first color and the second color are visually different.

In one or more embodiments, there is provided a kit for topical application comprising a colored or colorable topical composition comprising:
a foamable base composition, comprising:
   a. a flowable carrier composition;
   b. a color agent;
      i. wherein the color agent is in effective amount to effective to impart, increase, decrease or otherwise affect color of a foam produced from the foamable composition; and
      ii. wherein the color agent is one or more agents selected from the group consisting of a colored active agent, a colored excipient, a pigment, a dye, a colorant and a coloring agent;
   c. a propellant at a concentration of about 3% to about 25% by weight of the total composition;
   d. an aerosol container containing the base composition and propellant,
      1. wherein the base composition In one or more embodiments color; and
      2. wherein the foam comprising the colored topical composition has a second color upon dispensing form the aerosol container, and
      3. wherein the first color and the second color are visually different.

In one or more embodiments, there is provided use of a colored or colorable topical composition as a diagnostic, comprising:
a foamable base composition comprising:
   a. a flowable carrier composition;
   b. a color agent;
      i. wherein the color agent is effective to impart, increase, decrease or otherwise affect color of a foam produced from the foamable composition; and
      ii. wherein the color agent comprises a color indicator and optionally one or more agents selected from the group consisting of a colored active agent, a colored excipient, a pigment, a dye, a colorant and a coloring agent;
   c. a propellant at a concentration of about 3% to about 25% by weight of the total composition
      i. wherein the base composition has a first color; and
      ii. wherein the foam comprising the colored or colorable topical composition has a second color upon dispensing from an aerosol container, and
      iii. wherein the first color and the second color are visually different;
   d. applying the foam to a target surface or site
a foamable base composition comprising
   i. wherein the second color will change to a third color upon exposure to a parameter on or in the target surface or site to which the indictor is responsive, and
   ii. wherein the first color, the second color and the third color are each visually different.

There is also provided a formulation of any of the compositions described above wherein the composition is in a non foam state.

There is also provided a formulation of any of the compositions described above for use in the manufacture of a medicament.

### BRIEF DESCRIPTION OF THE DRAWINGS

FIG. 1 shows pictures of (1) the composition of Example 1 “as is” (prior to filling into the aerosol container and pressurizing; and (2) the foam produced from the same composition after filling into the aerosol container and pressurizing with 6% hydrocarbon propellant.

FIG. 2 shows pictures of (1) the composition of Example 3 “as is” (prior to filling into the aerosol container and pressurizing; and (2) the foam produced from the same composition after filling into the aerosol container and pressurizing with 6% hydrocarbon propellant.

FIG. 3 shows pictures of (1) the composition of Example 5 “as is” (prior to filling into the aerosol container and pressurizing; and (2) the foam produced from the same composition after filling into the aerosol container and pressurizing with 6% hydrocarbon propellant.

FIG. 4 shows shows pictures of the foam composition containing Methylene Blue into a model of a vaginal cavity.

FIG. 5 shows pictures of (1) composition CTR001 “as is” (prior to filling into the aerosol container and pressurizing; and (2) the foam produced from the same composition after filling into the aerosol container and pressurizing with 6% hydrocarbon propellant.

FIG. 6 shows pictures of (1) the composition of Example 10 “as is” (prior to filling into the aerosol container and pressurizing; and (2) the foam produced from the same composition after filling into the aerosol container and pressurizing with 8% hydrocarbon propellant.

FIGS. 7a and 7b show pictures of (1) the compositions 3 and 4 respectively of Example 11 “as is” (prior to filling into the aerosol container and pressurizing; and (2) the foam produced from the same composition after filling into the aerosol container and pressurizing with 8% hydrocarbon propellant.

FIGS. 8a and 8b show pictures of (1) the compositions 5 and 7c respectively of Example 12 “as is” (prior to filling into the aerosol container and pressurizing; and (2) the foam produced from the same composition after filling into the aerosol container and pressurizing with 8% hydrocarbon propellant.

FIGS. 9a and 9b show pictures of (1) the compositions 6A and 7A respectively of Example 11 “as is” (prior to filling into the aerosol container and pressurizing; and (2) the foam produced from the same composition after filling into the aerosol container and pressurizing with 8% hydrocarbon propellant.

FIG. 10 shows pictures of (1) the composition 2 of Example 14 “as is” (prior to filling into the aerosol container and pressurizing; and (2) the foam produced from the same composition after filling into the aerosol container and pressurizing with 8% hydrocarbon propellant.

FIG. 11 shows pictures of (1) the composition of Example 15 “as is” (prior to filling into the aerosol container and pressurizing; and (2) the foam produced from the same composition after filling into the aerosol container and pressurizing with 8% hydrocarbon propellant.

FIG. 12 shows pictures of (1) the composition 30 of Example 17 “as is” (prior to filling into the aerosol container and pressurizing; and (2) the foam produced from the same composition after filling into the aerosol container and pressurizing with 8% hydrocarbon propellant.

FIG. 13 shows pictures of (1) the composition 9 of Example 18 “as is” (prior to filling into the aerosol container and pressurizing; and (2) the foam produced from the same composition after filling into the aerosol container and pressurizing with 8% hydrocarbon propellant.

FIGS. 14a and 14b show pictures prior to and after conversion to nano emulsion size of (1) the composition 10 of Example 19 “as is” (prior to filling into the aerosol container and pressurizing; and (2) the foam produced from the same composition after filling into the aerosol container and pressurizing with 8% hydrocarbon propellant.

FIG. 15 shows pictures of (1) the composition 12 of Example 20 “as is” (prior to filling into the aerosol container and pressurizing; and (2) the foam produced from the same composition after filling into the aerosol container and pressurizing with 8% hydrocarbon propellant.

### DETAILED DESCRIPTION

There is provided a composition for use as foamable vehicle composition and a safe and effective colored or colorable foamable cosmetic or pharmaceutical vehicle or composition.

It was discovered that incorporating a colored active agent in a foamable composition results in a product with a significant and visual change of color, when compared with the composition which is not foamed. Based on this discovery it is possible to develop and create a multitude of foams which can use color and the color change on foaming a composition:

1. improve patient compliance especially in children or sensitive patients;

2. to make a foam more visually attractive;

3. as an indicator the foam has been absorbed;

4. to distinguish between foams;

5. to determine that the foam has been evenly applied to a target area;

6. to indicate an area has been treated;

7. as a preliminary diagnostic;

8. to dye a target area; and

9. to reduce or minimize staining.

In one embodiment, the color decreases. In another, it increases, and, in a still further embodiment, it varies depending on one or more factors such as a change in light, heat, pH, chemical association or reaction, oxidation or reduction, an osmotic factor, the special orientation of the component of the composition or the elimination or reduction in one or more components, such as a volatile component.

Thus, according to one or more embodiments, the foamable carrier, includes:
a. a foamable carrier composition;
b. a colored active agent; and
c. a propellant at a concentration of about 3% to about 25% by weight of the total composition.

In one or more embodiments, there is provided a colored or colorable topical composition, comprising:
a. a foamable base composition comprising
   i. a flowable carrier composition;
   ii. a color agent;
      1. wherein the color agent is effective to impart, increase, decrease or otherwise affect color of a foam produced from the foamable composition and
      2. wherein the color agent is one or more agents selected from the group consisting of a colored active agent, colored indicator, a colored excipient, a pigment, a dye, a colorant and a coloring agent.
b. a propellant at a concentration of about 3% to about 25% by weight of the total composition
wherein the base composition has a first color; and
wherein the foam comprising the colored or colorable topical composition has a second color upon dispensing the topical composition from an aerosol container, and
wherein the first color and the second color are visually different.

In one or more embodiments, the color difference between the first and second color is a difference in one or more of intensity, luminance, lightness and hue.

In one or more embodiments, the color difference is about 1% to about 75% of one or more of the internationally recognized parameters for color of intensity, luminance, lightness and hue.

In one or more embodiments, the color difference is at least 5%.

In one or more embodiments, one or more of the color parameters have decreased.

In one or more embodiments, the color parameter that decreased is selected from the group consisting of intensity and lightness or both.

In one or more embodiments, the second color is off white.

In one or more embodiments, the flowable carrier composition comprises:
at least one carrier, selected from the group consisting of water, an alcohol, a polyol, a polyethylene glycol (PEG), a polar solvent and a hydrophobic carrier comprising an oil, a petrolatum, a silicone oil, a triglyceride and an ester of a fatty acid;
at least one stabilizing component, selected from the group consisting of:
a. a surface active agent;
b. a polymeric agent;
optionally a foam adjuvant agent, selected from the group consisting of a fatty alcohol having 15 or more carbons in their carbon chain; a fatty acid having 16 or more carbons in their carbon chain;
wherein the color active agent is sufficiently soluble in the carrier to give expression to the color and the composition is selected from the group consisting of a non aqueous composition, a substantially non aqueous composition or an aqueous composition.

In one or more embodiments, the composition further comprises a color modifying agent.

In one or more embodiments, the foamable composition comprises an aliphatic alcohol, water, a fatty alcohol and a surface active agent.

In one or more embodiments, the foamable composition is an emulsion, comprising water, a hydrophobic carrier, a surface-active agent and a polymeric agent wherein the emulsion is selected from the group consisting of a macro, a micro, and a nano, oil in water or a water in oil emulsion.

In one or more embodiments, the hydrophobic carrier is occlusive.

In one or more embodiments, the foamable composition is oleaginous.

In one or more embodiments, the composition includes more than 50% of a polar solvent

In one or more embodiments, the surface active agent is selected from the group consisting of a polysorbate, polyoxyethylene (20) sorbitan monostearate, polyoxyethylene (20) sorbitan monooleate, a polyoxyethylene fatty acid ester, myrj 45, myrj 49, myrj 52 and myrj 59, a polyoxyethylene alkylyl ether, polyoxyethylene cetyl ether, polyoxyethylene palmityl ether, polyethylene oxide hexadecyl ether, polyethylene glycol cetyl ether, brij 38, brij 52, brij 56 brij 72, brij 721 and brij w1, a sucrose ester, a partial ester of sorbitol, sorbitan monolaurate, sorbitan monolaurate a monoglyceride, a diglyceride, isoceteth-20, a sucrose ester, or selected from the group consisting of steareth 2, glyceryl monostearate/peg 100 stearate, glyceryl stearate, steareth-21, peg 40 stearate, polysorbate 60, polysorbate 80, sorbitan stearate, laureth 4, sorbitan monooleate, ceteareth 16 ceteareth 20, steareth 10, steareth 20, ceteth 20, macrogol cetostearyl ether, ceteth 2, peg-30 dipolyhydroxystearate, sucrose distearate, polyoxyethylene (100) stearate, peg 40 stearate, peg 100 stearate, laureth 4, cetomacrogol ether, cetearyl alcohol, cetearyl glucoside, oleyl alcohol, steareth-2, diisopropyl adipate, capric/caprilic triglicerides, polysorbate 20; polysorbate 80, montanov 68 (cetearyl alcohol (and) cetearyl glucoside.), simusol 165 (glyceryl stearate and peg-100 stearate). methyl glucose sequistearate, peg 30 dipolyhydroxystearate, sucrose stearic acid esters, sorbitan laureth, sorbitan stearate, polyglyceryl-10 laurate, epikuuron 80, span 80 and mixtures thereof. and wherein the polymeric agent is selected from the group consisting of locust bean gum, sodium alginate, sodium caseinate, egg albumin, gelatin agar, carrageenin gum, sodium alginate, xanthan gum, quince seed extract, tragacanth gum, guar gum, cationic guars, hydroxypropyl guar gum, starch, an amine-bearing polymer, chitosan, alginic acid, hyaluronic acid, a chemically modified starch, a carboxyvinyl polymer, polyvinylpyrrolidone, polyvinyl alcohol, a polyacrylic acid polymer, a polymethacrylic acid polymer, polyvinyl acetate, a polyvinyl chloride polymer, a polyvinylidene chloride polymer, methylcellulose, hydroxypropyl cellulose, hydroxypropyl methylcellulose, hydroxyethyl cellulose, hydroxy propylmethyl cellulose, methylhydroxyethylcellulose, methylhydroxypropylcellulose, hydroxyethylcarboxymethylcellulose, carboxymethyl cellulose, carboxymethylcellulose carboxymethylhydroxyethylcellulose, a cationic cellulose peg 1000, peg 4000, peg 6000 and peg 8000, Carbopol® 934, Carbopol® 940, Carbopo® 941, Carbopol® 980, Carbopol® 981. hydroxypropylcellulose and carbomer.

In one or more embodiments the colored active agent is selected from a chemically derived active agent and an extract, wherein the extract is from mineral, plant, or animal source.

In one or more embodiments, the colored active agents is selected from the group consisting of iodine, povidone Iodine, coal tar extract, hammamelis extract, tetracycline, minocycline, doxorubicin, ichthyol, sulfur, anthralin, camellia sinensis, grape vine leaf powder extract, permethrine, methylene blue, alkanna, beta carotene, rosmarinic acid and quercetin.

In one or more embodiments, the colored active agent is an extract from a source selected from angelica, calendula, celery, coltsfoot, comfrey, dandelion, jamaica dogwood, kava, marshmallow, prickly ash, northern prickly ash, southern senna, valerian, agrimony, aloe vera, alfalfa, artichoke, avens, bayberry, bloodroot, blue flag, bogbean, boldo, boneset, broom, buchu, burdock, burnet, calamus, calendula, cascara, centaury, cereus, chamomile, german chamomile, roman chamomile, cinnamon, clivers, cohosh, black, cohosh, blue, cola, corn silk, couchgrass, cowslip, damiana, devil's claw, drosera, echinacea, elder, elecampane, euphorbia, eyebright, figwort, frangula, fucus, fumitory, garlic, golden seal, gravel root, ground ivy, guaiacum, hawthorn, holy thistle, hops, horehound black, horehound white, horse chestnut hydrangea, ispaghula, juniper, lady's lipper, liferoot, lime flower, liquorice, lobelia, mate, meadowsweet, mistletoe, motherwort, myrrh, nettle, parsley, parsley piert, passionflower, pennyroyal, pilewort, plantain, pleurisy root, pokeroot, poplar, pulsatilla, queen's delight, raspberry, red clover, rosemary, sage, sarsaparilla, sassafras, scullcap, senega, shepherd's purse, skunk cabbage, slippery elm, squill, St. john's wort, stone root, tansy, thyme, uva-ursi, vervain, wild carrot, wild lettuce, willow, witch hazel, yarrow and yellow dock.

In one or more embodiments, the colored active agent is colored in its raw material state

In one or more embodiments, the colored active agent renders noticeable color to a semi-solid formulation upon inclusion in such formulation.

In one or more embodiments, the colored active agent is selected from the group consisting of herbal extracts, mineral extracts, animal extracts, acaricides, age spot and keratose removing agents, allergen, analgesics, local anesthetics, antiacne agents, antiallergic agents, antiaging agents, antibacterials, antibiotics, antiburn agents, anticancer agents, antidandruff agents, antidepressants, antidermatitis agents, antiedemics, antihistamines, antihelminths, antihyperkeratolyte agents, antiinflammatory agents, antiirritants, antimicrobials, antimycotics, antiproliferative agents, antioxidants, anti-wrinkle agents, antipruritics, antipsoriatic agents, antirosacea agents antiseborrheic agents, antiseptic, antiswelling agents, antiviral agents, antiyeast agents, astringents, topical cardiovascular agents, chemotherapeutic agents, corticosteroids, disinfectants, fungicides, hair growth regulators, immunosuppressants, immunoregulating agents, insecticides, insect repellents, keratolytic agents, lactams, metals, metal oxides, mitocides, neuropeptides, non-steroidal anti-inflammatory agents, oxidizing agents, pediculicides, photodynamic therapy agents, retinoids, sanatives, scabicides, self tanning agents, skin whitening agents, asoconstrictors, vasodilators, vitamins, vitamin D derivatives, wound healing agents and wart removers.

In one or more embodiments, the composition further comprises an additional component selected from the group consisting of an anti perspirant, an anti-static agent, a buffering agent, a bulking agent, a chelating agent, a colorant, a conditioner, a deodorant, a diluent, a dye, an emollient, fragrance, a humectant, an occlusive agent, a penetration enhancer, a perfuming agent, a permeation enhancer, a pH-adjusting agent, a preservative, a skin penetration enhancer, a sunscreen, a sun blocking agent, a sunless tanning agent, and a vitamins.

In one or more embodiments, the composition further comprises an additional therapeutic agent, selected from the group consisting of active herbal extracts, acaricides, age spot and keratose removing agents, allergen, analgesics, local anesthetics, antiacne agents, antiallergic agents, antiaging agents, antibacterials, antibiotics, antiburn agents, anticancer agents, antidandruff agents, antidepressants, antidermatitis agents, antiedemics, antihistamines, antihelminths, antihyperkeratolyte agents, antiinflammatory agents, antiirritants, antilipemics, antimicrobials, antimycotics, antiproliferative agents, antioxidants, anti-wrinkle agents, antipruritics, antipsoriatic agents, antirosacea agents antiseborrheic agents, antiseptic, antiswelling agents, antiviral agents, anti-yeast agents, astringents, topical cardiovascular agents, chemotherapeutic agents, corticosteroids, dicarboxylic acids, disinfectants, fungicides, hair growth regulators, hormones, hydroxy acids, immunosuppressants, immunoregulating agents, insecticides, insect repellents, keratolytic agents, lactams, metals, metal oxides, mitocides, neuropeptides, non-steroidal anti-inflammatory agents, oxidizing agents, pediculicides, photodynamic therapy agents, retinoids, sanatives, scabicides, self tanning agents, skin whitening agents, asoconstrictors, vasodilators, vitamins, vitamin D derivatives, wound healing agents and wart removers.

In one or more embodiments, there is provided a method of changing the color of a colored or colorable topical composition, comprising:
a. selecting a color agent, a flowable carrier composition a propellant and an aerosol canister;
b. preparing a colored foamable base composition of a first color, comprising:
   i. a flowable carrier composition; and
   ii. a color agent;
wherein the agent is effective to impart, increase, decrease or otherwise affect color of a foam produced from the foamable composition; and
wherein the agent is one or more agents selected from the group consisting of a colored active agent, a colored excipient, a pigment, a dye, a colorant and a coloring agent;
c. filling the foamable base composition in the aerosol container, closing the container having an aerosol valve, adding a propellant at a concentration of about 3% to about 25% by weight of the total composition; and
d. opening the aerosol valve to release foam of a second color;
wherein the first color and the second color are visually different.

In one or more embodiments, the method further comprises selecting a color modifying agent and preparing a foamable composition further comprising a color modifying agent.

In one or more embodiments, the method further comprises selecting a color indicator, preparing a foamable composition further comprising a color indictor and applying the foam to a target surface wherein the second color will change to a third color upon exposure to a parameter on or in the target surface to which the indicator is responsive and wherein the first color, the second color and the third color are each visually different In one or more embodiments, there is provided a method of treating a disorder of a mammalian subject to achieve an improved compliance, comprising:
administering a colored or colorable topical composition to a target site, the colored topical composition, comprising:
a. a foamable base composition comprising
   i. a flowable carrier composition;
   ii. a color agent
      i. wherein the color agent is effective to impart, increase, decrease or otherwise affect color of a foam produced from the foamable composition and
      ii. wherein the color agent is one or more agents selected from the group consisting of a colored active agent, a colored indicator, a colored excipient, a pigment, a dye, a colorant and a coloring agent and;
      iii. wherein the color agent comprises at least an effective amount of active agent;
b. a propellant at a concentration of about 3% to about 25% by weight of the total composition
wherein the base composition has a first color; and
wherein the colored topical composition has a second color after it has been dispensed from an aerosol container, and
wherein the first color and the second color are visually different.

In one or more embodiments, the target site is selected from the group consisting of the skin, a body cavity, a mucosal surface, the nose, the mouth, the eye, the ear canal, the respiratory system, the vagina and the rectum.

In one or more embodiments, the disorder is selected from the group consisting of dermatological pain, dermatological inflammation, acne, acne vulgaris, inflammatory acne, non-inflammatory acne, acne fulminans, nodular papulopustular acne, acne conglobata, dermatitis, bacterial skin infections, fungal skin infections, viral skin infections, parasitic skin infections, skin neoplasia, skin neoplasms, pruritis, cellulitis, acute lymphangitis, lymphadenitis, erysipelas, cutaneous abscesses, necrotizing subcutaneous infections, scalded skin syndrome, folliculitis, furuncles, hidradenitis suppurativa, carbuncles, paronychial infections, rashes, erythrasma, impetigo, eethyma, yeast skin infections, warts, molluscum contagiosum, trauma or injury to the skin, post-operative or post-surgical skin conditions, scabies, pediculosis, creeping eruption, eczemas, psoriasis, pityriasis rosea, lichen planus, pityriasis rubra pilaris, edematous, erythema multiforme, erythema nodosum, grannuloma annulare, epidermal necrolysis, sunburn, photosensitivity, pemphigus, bullous pemphigoid, dermatitis herpetiformis, keratosis pilaris, callouses, corns, ichthyosis, skin ulcers, ischemic necrosis, miliaria, hyperhidrosis, moles, Kaposi's sarcoma, melanoma, malignant melanoma, basal cell carcinoma, squamous cell carcinoma, poison ivy, poison oak, contact dermatitis, atopic dermatitis, rosacea, purpura, moniliasis, candidiasis, baldness, alopecia, Behcet's syndrome, cholesteatoma, Dercum disease, ectodermal dysplasia, gustatory sweating, nail patella syndrome, lupus, hives, hair loss, Hailey-Hailey disease, chemical or thermal skin burns, scieroderma, aging skin, wrinkles, sun spots, necrotizing fasciitis, necrotizing myositis, gangrene, scarring, and vitiligo, chlamydia infection, gonorrhea infection, hepatitis B, herpes, HIV/AIDS, human papillomavirus (HPV), genital warts, bacterial vaginosis, candidiasis, chancroid, granuloma Inguinale, lymphogranloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, pelvic inflammation, endometritis, salpingitis, oophoritis, genital cancer, cancer of the cervix, cancer of the vulva, cancer of the vagina, vaginal dryness, dyspareunia, anal and rectal disease, anal abscess/fistula, anal cancer, anal fissure, anal warts, Crohn's disease, hemorrhoids, anal itch, pruritus ani, fecal incontinence, constipation, polyps of the colon and rectum; and wherein the active agent is suitable for treating said disorder.

In one or more embodiments, there is provided a kit for topical application comprising a colored or colorable topical composition comprising:
a. foamable base composition, comprising:
   a. a flowable carrier composition;
   b. a color agent;
      i. wherein the color agent is in effective amount to effective to impart, increase, decrease or otherwise affect color of a foam produced from the foamable composition; and
      ii. wherein the color agent is one or more agents selected from the group consisting of a colored active agent, a colored excipient, a pigment, a dye, a colorant and a coloring agent;
   c. a propellant at a concentration of about 3% to about 25% by weight of the total composition;
   d. an aerosol container containing the base composition and propellant,
      1. wherein the base composition In one or more embodiments color; and
      2. wherein the foam comprising the colored topical composition has a second color upon dispensing form the aerosol container, and
      3. wherein the first color and the second color are visually different.

In one or more embodiments, there is provided use of a colored or colorable topical composition as a diagnostic, comprising:
a foamable base composition comprising
   a. a flowable carrier composition;
   b. a color agent;
      i. wherein the color agent is effective to impart, increase, decrease or otherwise affect color of a foam produced from the foamable composition; and
      ii. wherein the color agent comprises a color indicator and optionally one or more agents selected from the group consisting of a colored active agent, a colored excipient, a pigment, a dye, a colorant and a coloring agent;
   c. a propellant at a concentration of about 3% to about 25% by weight of the total composition,
      i. wherein the base composition has a first color; and
      ii. wherein the foam comprising the colored or colorable topical composition has a second color upon dispensing from an aerosol container, and
      iii. wherein the first color and the second color are visually different
   d. applying the foam to a target surface or site,
      i. wherein the second color will change to a third color upon exposure to a parameter on or in the target surface or site to which the indictor is responsive, and
      ii. wherein the first color, the second color and the third color are each visually different.

In accordance with one or more embodiments, there is also provided a colored or colorable topical composition wherein the coloring agent is an active agent.

In accordance with one or more embodiments, the flowable carrier composition, comprises at least one carrier, selected from the group consisting of water, an oil, a silicone oil, an alcohol, a polyol, a polyethylene glycol (PEG) and a solvent.

In accordance with one or more embodiments, the foamable composition further comprises at least one component, selected from the group consisting of:

a. a surface active agent; and

b. a polymeric agent.

In accordance with one or more embodiments the colored or colorable topical composition further comprises a color modifying agent.

In an exemplary embodiment, the foamable colored or colorable topical composition is an aqueous composition, containing water and further comprises a surface active agent.

In an exemplary embodiment, the foamable colored or colorable topical composition comprises an aliphatic alcohol, water, a fatty alcohol and a surface active agent.

In an exemplary embodiment, the foamable colored or colorable topical composition is an emulsion, comprising water, a hydrophobic solvent, a surface-active agent and a polymeric agent. Optionally, the emulsion-type foamable composition further contains a foam adjuvant

In certain embodiments, the emulsion is an oil in water emulsion, while in additional embodiments the emulsion is a water in oil emulsion.

In certain embodiments, the hydrophobic carrier is an oil. Exemplary oils include mineral oil, silicone oil, a triglyceride and an ester of a fatty acid. In certain embodiments, the hydrophobic solvent is occlusive, such as petrolatum, while in other embodiments the hydrophobic carrier in non-occlusive.

In an exemplary embodiment, the foamable colored or colorable topical composition is an oleaginous foamable composition, including at least one solvent selected from a hydrophobic solvent, a silicone oil, an emollient, a polar solvent and mixtures thereof, wherein the solvent is present at a concentration of about 70% to about 96.5% by weight of the total composition, at least a non-ionic surface-active agent and at least one polymeric agent.

In an exemplary embodiment, the foamable colored or colorable topical composition includes more than 50% of a polar solvent (as used herein, the term “polar solvent” shall mean a material that produces a uniform, clear or hazy, mixture when combined with at least a weight equivalent of water), a surface-active agent and a polymeric agent. In certain embodiments the foamable composition is substantially water free, while in additional embodiments the foamable composition contains up to 25% water.

In one or more embodiments, the composition is substantially water-free.

### Foam Adjuvant

Optionally, the foamable vehicle further includes a foam adjuvant selected from the group consisting of a fatty alcohol having 15 or more carbons in their carbon chain; a fatty acid having 16 or more carbons in their carbon chain; fatty alcohols, derived from beeswax and including a mixture of alcohols, a majority of which has at least 20 carbon atoms in their carbon chain; a fatty alcohol having at least one double bond; a fatty acid having at least one double bond; a branched fatty alcohol; a branched fatty acid and a fatty acid substituted with a hydroxyl group.

### Hydrophobic Carrier

A “hydrophobic solvent” as used herein refers to a material having solubility in distilled water at ambient temperature of less than about 1 gm per 100 mL, more preferable less than about 0.5 gm per 100 mL, and most preferably less than about 0.1 gm per 100 mL.

In one or more embodiments, the hydrophobic organic carrier is an oil, such as mineral oil, triglycerides, capric/caprylic triglyceride, alkyl esters of fatty acids such as isopropyl palmitate, isopropyl isostearate, diisopropyl adipate, diisopropyl dimerate, maleated soybean oil, octyl palmitate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, ricinoleate, isopropyl lanolate, pentaerythrityl tetrastearate, neopentylglycol dicaprylate/dicaprate, isononyl isononanoate, isotridecyl isononanoate, myristyl myristate, triisocetyl citrate, octyl dodecanol, unsaturated or polyunsaturated oils, such as olive oil, corn oil, soybean oil, canola oil, cottonseed oil, coconut oil, sesame oil, sunflower oil, borage seed oil, syzigium aromaticum oil, hempseed oil, herring oil, cod-liver oil, salmon oil, flaxseed oil, wheat germ oil, evening primrose oils; essential oils; and silicone oils, such as dimethicone, cyclomethicone, polyalkyl siloxane, polyaryl siloxane, polyalkylaryl siloxane, a polyether siloxane copolymer and a poly(dimethylsiloxane)-(diphenyl-siloxane) copolymer.

### Surface Active Agent

The composition further contains a surface-active agent. Surface-active agents (also termed “surfactants”) include any agent linking oil and water in the composition, in the form of emulsion. A surfactant's hydrophilic/lipophilic balance (HLB) describes the emulsifier's affinity toward water or oil. HLB is defined for non-ionic surfactants. The HLB scale ranges from 1 (totally lipophilic) to 20 (totally hydrophilic), with 10 representing an equal balance of both characteristics. Lipophilic emulsifiers form water-in-oil (w/o) emulsions; hydrophilic surfactants form oil-in-water (o/w) emulsions. The HLB of a blend of two emulsifiers equals the weight fraction of emulsifier A times its HLB value plus the weight fraction of emulsifier B times its HLB value (weighted average). In many cases a single surfactant may suffice. In other cases a combination of two or more surfactants is desired. Reference to a surfactant in the specification can also apply to a combination of surfactants or a surfactant system. As will be appreciated by a person skilled in the art which surfactant or surfactant system is more appropriate is related to the vehicle and intended purpose. In general terms a combination of surfactants is usually preferable where the vehicle is an emulsion. In an emulsion environment a combination of surfactants can be significant in producing breakable forms of good quality. It has been further discovered that the generally thought considerations for HLB values for selecting a surfactant or surfactant combination are not always binding for emulsions and that good quality foams can be produced with a surfactant or surfactant combination both where the HLB values are in or towards the lipophilic side of the scale and where the HLB values are in or towards the hydrophilic side of the scale. Surfactants also play a role in foam formation where the foamable formulation is a single phase composition.

According to one or more embodiments, the composition contains a single surface active agent having an HLB value between about 2 and 9, or more than one surface active agent and the weighted average of their HLB values is between about 2 and about 9. Lower HLB values may in certain embodiments be more applicable to water in oil emulsions.

According to one or more embodiments, the composition contains a single surface active agent having an HLB value between about 7 and 14, or more than one surface active agent and the weighted average of their HLB values is between about 7 and about 14. Mid range HLB values may in certain embodiments be more suitable for oil in water emulsions.

According to one or more other embodiments, the composition contains a single surface active agent having an HLB value between about 9 and about 19, or more than one surface active agent and the weighted average of their HLB values is between about 9 and about 19. In a waterless or substantially waterless environment a wide range of HLB values may be suitable.

Preferably, the composition contains a non-ionic surfactant. Non-limiting examples of possible non-ionic surfactants include a polysorbate, polyoxyethylene (20) sorbitan monostearate, polyoxyethylene (20) sorbitan monooleate, a polyoxyethylene fatty acid ester, Myrj 45, Myrj 49, Myrj 52 and Myrj 59; a polyoxyethylene alkyl ether, polyoxyethylene cetyl ether, polyoxyethylene palmityl ether, polyethylene oxide hexadecyl ether, polyethylene glycol cetyl ether, steareths such as steareth 2, brij 21, brij 721, brij 38, brij 52, brij 56 and brij W1, a sucrose ester, a partial ester of sorbitol and its anhydrides, sorbitan monolaurate, sorbitan monolaurate, a monoglyceride, a diglyceride, isoceteth-20 and mono-, di- and tri-esters of sucrose with fatty acids. In certain embodiments, suitable sucrose esters include those having high monoester content, which have higher HLB values.

In certain embodiments with DCA esters as emollient, surfactants are selected which can provide a close packed surfactant layer separating the oil and water phases. To achieve such objectives combinations of at least two surfactants are selected. Preferably, they should be complex emulgators and more preferably they should both be of a similar molecular type. For example, a pair of ethers, like steareth 2 and steareth 21, or a pair of esters for example, PEG-40 stearate and polysorbate 80. In Certain circumstances POE esters cannot be used and a combination of sorbitan laurate and sorbitan stearate or a combination of sucrose stearic acid ester mixtures and sodium laurate may be used. All these combinations due to heir versatility and strength may also be used satisfactorily and effectively with solutions of DCA's and with solid/crystalline suspensions, although the amounts and proportion may be varied according to the formulation and its objectives as will be appreciated by a man of the art.

It has been discovered also that by using a derivatized hydrophilic polymer with hydrophobic alkyl moieties as a polymeric emulsifier such as permulen it is possible to stabilize the emulsion better about or at the region of phase reversal tension. Other types of derivatized polymers like silicone copolymers, derivatized starch [Aluminum Starch Octenylsuccinate (ASOS)]/[DRY-FLO AF Starch], and derivatized dexrin may also a similar stabilizing effect.

A series of dextrin derivative surfactants prepared by the reaction of the propylene glycol polyglucosides with a hydrophobic oxirane-containing material of the glycidyl ether are highly biodegradable. [Hong-Rong Wang and Keng-Ming Chen, Colloids and Surfaces A: Physicochemical and Engineering Aspects Volume 281, Issues 1-3, 15 Jun. 2006, Pages 190-193].

Non-limiting examples of non-ionic surfactants that have HLB of about 7 to about 12 include steareth 2 (HLB˜4.9); glyceryl monostearate/PEG 100 stearate (Av HLB˜11.2); stearate Laureth 4 (HLB˜9.7) and cetomacrogol ether (e.g., polyethylene glycol 1000 monocetyl ether).

Non-limiting examples of preferred surfactants, which have a HLB of 4-19 are set out in the Table below:

| Surfactant | HLB |
|---|---|
| steareth 2 | ~4.9 |
| glyceryl monostearate/PEG 100 stearate | Av ~11.2 |
| Glyceryl Stearate | ~4 |
| Steareth-21 | ~15.5 |
| peg 40 stearate | ~16.9 |
| polysorbate 80 | ~15 |
| sorbitan stearate | ~4.7 |
| laureth 4 | ~9.7 |
| Sorbitan monooleate (span 80) | ~4.3 |
| ceteareth 20 | ~15.7 |
| steareth 20 | ~15.3 |
| ceteth 20 | ~15.7 |
| Macrogol Cetostearyl Ether | ~15.7 |
| ceteth 2 (Lipocol C-2) | ~5.3 |
| PEG-30 Dipolyhydroxystearate | ~5.5 |
| sucrose distearate (Sisterna SP30) | ~6 |
| polyoxyethylene (100) stearate | ~18.8 |

More exemplary stabilizing surfactants which may be suitable for use in the present invention are found below.

### PEG-Fatty Acid Monoester Surfactants

| Chemical name | Product example name | HLB |
|---|---|---|
| PEG-30 stearate | Myrj 51 | >10 |
| PEG-40 laurate | Crodet L40 (Croda) | 17.9 |
| PEG-40 oleate | Crodet O40 (Croda) | 17.4 |
| PEG-45 stearate | Nikkol MYS-45 (Nikko) | 18 |
| PEG-50 stearate | Myrj 53 | >10 |
| PEG-100 stearate | Myrj 59, Arlacel 165 (ICI) | 19 |

### PEG-Fatty Acid Diester Surfactants:

| Chemical name | Product example name | HLB |
|---|---|---|
| PEG-4 dilaurate | Mapeg .RTM. 200 DL (PPG), | 7 |
| | Kessco .RTM.PEG 200 DL | |
| | (Stepan), LIPOPEG 2-DL (Lipo | |
| | Chem.) | |
| PEG-4 | distearate Kessco .RTM. 200 | 5 |
| | DS (Stepan.sub) | |
| PEG-32 dioleate | Kessco .RTM. PEG 1540 DO | 15 |
| | (Stepan) | |
| PEG-400 dioleate | Cithrol 4DO series (Croda) | >10 |
| PEG-400 disterate | Cithrol 4DS series (Croda) | >10 |
| PEG-20 glyceryl oleate | Tagat .RTM. O (Goldschmidt) | >10 |

### Transesterification Products of Oils and Alcohols

| Chemical name | Product example name | HLB |
|---|---|---|
| PEG-30 castor oil | Emalex C-30 (Nihon Emulsion) | 11 |
| PEG-40 hydrogenated | Cremophor RH 40 (BASF), | 13 |
| castor oil | Croduret (Croda), Emulgin | |
| | HRE 40 (Henkel) | |

### Polyglycerized Fatty Acids, Such as:

| Chemical name | Product example name | LB |
|---|---|---|
| Polyglyceryl-6 dioleate | Caprol .RTM. 6G20 (ABITEC); | 8.5 |
| | PGO-62 (Calgene), PLUROL | |
| | OLEIQUE CC 497 | |
| | (Gattefosse)Hodag | |

### PEG-Sorbitan Fatty Acid Esters

| Chemical name | Product example name | HLB |
|---|---|---|
| PEG-20 sorbitan | Tween-20 (Atlas/ICI), Crillet 1 | 17 |
| monolaurate | (Croda), DACOL MLS 20 | |
| | (Condea) | |
| PEG-20 sorbitan | Tween 40 (Atlas/ICI), Crillet 2 | 16 |
| Monopalmitate | (Croda) | |
| PEG-20 sorbitan | Tween-60 (Atlas/ICI), Crillet 3 | 15 |
| monostearate | (Croda) | |
| PEG-20 sorbitan | Tween-80 (Atlas/ICI), Crillet 4 | 15 |
| monooleate | (Croda) | |

### Polyethylene Glycol Alkyl Ethers

| Chemical name | Product example name | HLB |
|---|---|---|
| PEG-2 oleyl ether | oleth-2 Brij 92/93 (Atlas/ICI) | 4.9 |
| PEG-3 oleyl ether | oleth-3 Volpo 3 (Croda) | <10 |
| PEG-5 oleyl ether | oleth-5 Volpo 5 (Croda) | <10 |
| PEG-10 oleyl ether | oleth-10 Volpo 10 (Croda), Brij | 12 |
| | 96/97 (Atlas/ICI) | |
| PEG-20 oleyl ether | oleth-20 Volpo 20 (Croda), Brij | 15 |
| | 98/99 (Atlas/ICI) | |
| PEG-4 lauryl ether | laureth-4Brij 30 (Atlas/ICI) | 9.7 |
| PEG-23 lauryl ether | laureth-23Brij 35 (Atlas/ICI) | 17 |
| PEG-10 stearyl ether | Brij 76 (ICI) | 12 |
| PEG-2 cetyl ether | Brij 52 (ICI) | 5.3 |

### Sugar Ester Surfactants

| Chemical name | Product example name | HLB |
|---|---|---|
| Sucrose distearate | Sisterna SP50, Surfope 1811 | 11 |

### Sorbitan Fatty Acid Ester Surfactants

| Chemical name | Product example name | HLB |
|---|---|---|
| Sorbitan monolaurate | Span-20 (Atlas/ICI), Crill 1 | 8.6 |
| | (Croda), Arlacel 20 (ICI) | |
| Sorbitan monopalmitate | Span-40 (Atlas/ICI), Crill 2 | 6.7 |
| | (Croda), Nikkol SP-10 (Nikko) | |
| Sorbitan monooleate | Span-80 (Atlas/ICI), Crill 4 | 4.3 |
| | (Croda), Crill 50 (Croda) | |
| Sorbitan monostearate | Span-60 (Atlas/ICI), Crill 3 | 4.7 |
| | (Croda), Nikkol SS-10 (Nikko) | |

In one or more embodiments, the surface active agent is a complex emulgator in which the combination of two or more surface active agents can be more effective than a single surfactant and provides a more stable emulsion or improved foam quality than a single surfactant. For example and by way of non-limiting explanation it has been found that by choosing say two surfactants, one hydrophobic and the other hydrophilic the combination can produce a more stable emulsion than a single surfactant. Preferably, the complex emulgator comprises a combination of surfactants wherein there is a difference of about 4 or more units between the HLB values of the two surfactants or there is a significant difference in the chemical nature or structure of the two or more surfactants.

Specific non limiting examples of surfactant systems are, combinations of polyoxyethylene alkyl ethers, such as Brij 59/Brij 10; Brij 52/Brij 10; Steareth 2/Steareth 20; Steareth 2/Steareth 21 (Brij 72/Brij 721); combinations of polyoxyethylene stearates such as Myrj 52/Myrj 59; combinations of sucrose esters, such as Surphope 1816/Surphope 1807; combinations of sorbitan esters, such as Span 20/Span 80; Span 20/Span 60; combinations of sucrose esters and sorbitan esters, such as Surphope 1811 and Span 60; combinations of liquid polysorbate detergents and PEG compounds, such as Tween 80/PEG-40 stearate; methyl glucaso sequistearate; polymeric emulsifiers, such as Permulen (TR1 or TR2); liquid crystal systems, such as Arlatone (2121), Stepan (Mild RM1), Nikomulese (41) and Montanov (68) and the like.

In certain embodiments, the surfactant is preferably one or more of the following: a combination of steareth-2 and steareth-21 on their own or in combination with glyceryl monostearate (GMS); in certain other embodiments the surfactant is a combination of polysorbate 80 and PEG-40 stearate. In certain other embodiments the surfactant is a combination of glyceryl monostearate/PEG 100 stearate. In certain other embodiments the surfactant is a combination of two or more of stearate 21, PEG 40 stearate, and polysorbate 80. In certain other embodiments the surfactant is a combination of two or more of laureth 4, span80, and polysorbate 80. In certain other embodiments the surfactant is a combination of two or more of GMS and ceteareth. In certain other embodiments the surfactant is a combination of two or more of steareth 21, ceteareth 20, ceteth 2 and laureth 4 In certain other embodiments the surfactant is a combination of ceteareth 20 and polysorbate 40 stearate. In certain other embodiments the surfactant is a combination of span 60 and GMS.

In certain other embodiments, the surfactant is one or more of sucrose stearic acid esters, sorbitan laureth, and sorbitan stearate.

In one or more embodiments, the stability of the composition can be improved when a combination of at least one non-ionic surfactant having HLB of less than 9 and at least one non-ionic surfactant having HLB of equal or more than 9 is employed. The ratio between the at least one non-ionic surfactant having HLB of less than 9 and the at least one non-ionic surfactant having HLB of equal or more than 9, is between 1:8 and 8:1, or at a ratio of 4:1 to 1:4. The resultant HLB of such a blend of at least two emulsifiers is preferably between about 9 and about 14.

Thus, in an exemplary embodiment, a combination of at least one non-ionic surfactant having HLB of less than 9 and at least one non-ionic surfactant having HLB of equal or more than 9 is employed, at a ratio of between 1:8 and 8:1, or at a ratio of 4:1 to 1:4, wherein the HLB of the combination of emulsifiers is preferably between about 5 and about 18.

In certain cases, the surface active agent is selected from the group of cationic, zwitterionic, amphoteric and ampholytic surfactants, such as sodium methyl cocoyl taurate, sodium methyl oleoyl taurate, sodium lauryl sulfate, triethanolamine lauryl sulfate and betaines.

Many amphiphilic molecules can show lyotropic liquid-crystalline phase sequences depending on the volume balances between the hydrophilic part and hydrophobic part. These structures are formed through the micro-phase segregation of two Many amphiphilic molecules can show lyotropic liquid-crystalline phase sequences depending on the volume balances between the hydrophilic part and hydrophobic part. These structures are formed through the micro-phase segregation of two incompatible components on a nanometer scale. Soap is an everyday example of a lyotropic liquid crystal. Certain types of surfactants tend to form lyotropic liquid crystals in emulsions interface (oil-in-water) and exert a stabilizing effect

In one or more embodiments, the surfactant is a surfactant or surfactant combination is capable of or which tends to form liquid crystals. Surfactants which tend to form liquid crystals may improve the quality of foams. Non limiting examples of surfactants with postulated tendency to form interfacial liquid crystals are: phospholipids, alkyl glucosides, sucrose esters, sorbitan esters.

In one or more embodiments, the at least one surface active agent is liquid.

In one or more embodiments the at least one surface active agent is solid, semi solid or waxy.

It should be noted that HLB values may not be so applicable to non ionic surfactants, for example, with liquid crystals or with silicones. Also HLB values may be of lesser significance in a waterless or substantially non-aqueous environment.

In one or more embodiments, the surfactant can be, a surfactant system comprising of a surfactant and a co surfactant, a waxy emulsifier, a liquid crystal emulsifier, an emulsifier which is solid or semi solid at room temperature and pressure, or combinations of two or more agents in an appropriate proportion as will be appreciated a person skilled in the art. Where a solid or semi solid emulsifier combination is used it can also comprise a solid or semi solid emulsifier and a liquid emulsifier.

In one or more embodiments, the surface-active agent includes at least one non-ionic surfactant. Ionic surfactants are known to be irritants. Therefore, non-ionic surfactants are preferred in applications including sensitive tissue such as found in most mucosal tissues, especially when they are infected or inflamed. Non-ionic surfactants alone can provide formulations and foams of good or excellent quality in the carriers and compositions.

Thus, in a preferred embodiment, the surface active agent, the composition contains a non-ionic surfactant. In another preferred embodiment the composition includes a mixture of non-ionic surfactants as the sole surface active agent. Yet, in additional embodiments, the foamable composition includes a mixture of at least one non-ionic surfactant and at least one ionic surfactant in a ratio in the range of about 100:1 to 6:1. In one or more embodiments, the non-ionic to ionic surfactant ratio is greater than about 6:1, or greater than about 8:1; or greater than about 14:1, or greater than about 16:1, or greater than about 20:1. In further embodiments, surface active agent comprises a combination of a non-ionic surfactant and an ionic surfactant, at a ratio of between 1:1 and 20:1

In one or more embodiments, a combination of a non-ionic surfactant and an ionic surfactant (such as sodium lauryl sulphate and cocamidopropylbetaine) is employed, at a ratio of between 1:1 and 20:1, or at a ratio of 4:1 to 10:1; for example, about 1:1, about 4:1, about 8:1, about 12:1, about 16:1 and about 20:1 or ata ratio of 4:1 to 10:1, for example, about 4:1, about 6:1, about 8:1 and about 10:1.

In selecting a suitable surfactant or combination thereof it should be borne in mind that the upper amount of surfactant that may be used may be limited by the shakability of the composition. If the surfactant is non liquid, it can make the formulation to viscous or solid. This can be particularly significant if the formulation has high molecular weight, e.g., a high molecular weight PEG or polymeric agents or petroleum or if the surfactants are large. Solvents and polymeric agents which have high molecular weight and are very viscous or solid or waxy (e.g., Peg 1500, 2000, etc. or petrolatum) can exacerbate the effect of a waxy or solid surfactant on shakability or flowability In general terms, as the amount of non-liquid surfactant is increased the shakability of the formulation reduces until a limitation point is reached where the formulation becomes non shakable and unsuitable. Thus in one embodiment, an effective amount of surfactant may be used provided the formulation remains shakable. In other certain exceptional embodiments the upper limit may be determined by flowability such as in circumstances where the composition is marginally or apparently non-shakable. The formulation is sufficiently flowable to be able to flow through an actuator valve and be released and still expand to form a good quality foam.

In certain embodiments, the amount of surfactant or combination of surfactants is between about 0.05% to about 20%; between about 0.05% to about 15%. or between about 0.05% to about 10%. In a preferred embodiment the concentration of surface active agent is between about 0.2% and about 8%. In a more preferred embodiment the concentration of surface active agent is between about 1% and about 6%.

In some embodiments, it is desirable that the surface active agent does not contain a polyoxyethylene (POE) moiety, such as polysorbate surfactants, POE fatty acid esters, and POE alkyl ethers, because the active agent is incompatible with such surface active agents. For example, the active agent pimecrolimus is not stable the presence of POE moieties, yet benefits greatly from the use of dicarboxylic esters as penetration enhancers. In such cases, alternative surface active agents are employed. In an exemplary manner, POE—free surfactants include non-ethoxylated sorbitan esters, such as sorbitan monopalmitate, sorbitan monostearate, sorbitan tristearate, sorbitan monooleate, sorbitan trioleate, sorbitan monolaurate and sorbitan sesquioleate; glycerol fatty acid esters, such as glycerol monostearate and glycerol monooleate; mono-, di- and tri-esters of sucrose with fatty acids (sucrose esters), sucrose stearate, sucrose distearate sucrose palmitate and sucrose laurate; and alkyl polyglycosides, such as lauryl diglucoside.

If the composition as formulated is a substantially non shakable composition it is nevertheless possible as an exception in the scope for the formulation to be flowable to a sufficient degree to be able to flow through an actuator valve and be released and still expand to form a good quality foam. This surprising and unusual exception may be due one or more of a number of factors such as the high viscosity, the softness, the lack of crystals, the pseudoplastic or semi pseudo plastic nature of the composition and the dissolution of the propellant into the composition.

In one or more embodiments, the surface-active agent includes mono-, di- and tri-esters of sucrose with fatty acids (sucrose esters), prepared from sucrose and esters of fatty acids or by extraction from sucro-glycerides. Suitable sucrose esters include those having high monoester content, which have higher HLB values.

### Polymeric Agent

The composition contains a polymeric agent selected from the group consisting of a bioadhesive agent, a gelling agent, a film forming agent and a phase change agent. A polymeric agent enhances the creation of foam having fine bubble structure, which does not readily collapse upon release from the pressurized aerosol can. The polymeric agent serves to stabilize the foam composition and to control drug residence in the target organ.

Exemplary polymeric agents include, in a non-limiting manner, naturally-occurring polymeric materials, such as locust bean gum, sodium alginate, sodium caseinate, egg albumin, gelatin agar, carrageenin gum, sodium alginate, xanthan gum, quince seed extract, tragacanth gum, guar gum, cationic guars, hydroxypropyl guar gum, starch, amine-bearing polymers such as chitosan; acidic polymers obtainable from natural sources, such as alginic acid and hyaluronic acid; chemically modified starches and the like, carboxyvinyl polymers, polyvinylpyrrolidone, polyvinyl alcohol, polyacrylic acid polymers, polymethacrylic acid polymers, polyvinyl acetate polymers, polyvinyl chloride polymers, polyvinylidene chloride polymers and the like.

Additional exemplary polymeric agents include semi-synthetic polymeric materials such as cellulose ethers, such as methylcellulose, hydroxypropyl cellulose, hydroxypropyl methylcellulose, hydroxyethyl cellulose, hydroxy propylmethyl cellulose, methylhydroxyethylcellulose, methylhydroxypropylcellulose, hydroxyethylcarboxymethylcellulose, carboxymethyl cellulose, carboxymethylcellulose carboxymethylhydroxyethylcellulose, and cationic celluloses, carbomer (homopolymer of acrylic acid is crosslinked with an allyl ether pentaerythritol, an allyl ether of sucrose, or an allyl ether of propylene, such as Carbopol® 934, Carbopol® 940, Carbopo® 941, Carbopol® 980 and Carbopol® 981, pemulen and aluminum starch octenylsuccinate (ASOS). Polyethylene glycol, having molecular weight of 1000 or more (e.g., PEG 1,000, PEG 4,000, PEG 6,000 and PEG 10,000) also have gelling capacity and while they are considered herein as “secondary polar solvents”, as detailed herein, they are also considered polymeric agents.

In one or more embodiments, the polymeric agents have emulsifying properties. In certain preferred embodiments the polymeric agent is a derivatized hydrophilic polymer with hydrophobic alkyl moieties Other types that may also a similar stabilizing effect are silicone copolymers and derivatized starch ASOS.

Mixtures of the above polymeric agents are contemplated.

The concentration of the polymeric agent should be selected so that the composition, after filling into aerosol canisters, is flowable, and can be shaken in the canister. In one or more embodiments, the concentration of the polymeric agent is selected such that the viscosity of the composition, prior to filling of the composition into aerosol canisters, is about less than 15000 CPs, preferably less than 12,000 CPs, and more preferably, less than 10,000 CPs.

### Phase Inversion and Tension

Phase inversion is a factor in the preparation and stabilization of emulsions and can be both an aid and a detriment. Phase inversion involves the change of emulsion type from o/w to w/o or vice versa. Prior to phase inversion occurring there is a tension in the emulsion which if destabilized or driven will lead to phase inversion and if controlled or ameliorated or dissipated will result in a more stable emulsion. The occurrence of phase inversion during preparation can be a sign of instability. If controlled, it can result in a finer product but if due to other factors after the emulsion was prepared it can cause problems. Inversion can occur by for example adding calcium chloride to an o/w emulsion stabilized with sodium stearate to form calcium stearate. Inversion can also occur as the product of changes to the phase-volume ratio. For example if a small amount of water is added to surfactant mixed with oil and agitated a w/o emulsion is formed As the amount of water added is gradually increased a point will be reached where the water and emulsifier envelop the oil as small droplets to form an o/w emulsion. The amount of each ingredient including the surfactants will have their part to play in the phenomenon.

According to one or more embodiments, phase inversion can affect the dispersion of light in the formulation and foam and which in certain aspects can result in a potentiated color effect and in certain other aspects result in an ameliorated color effect.

### Substantially Alcohol-Free

According to one or more embodiments, the foamable composition is substantially alcohol-free, i.e., free of short chain alcohols. Short chain alcohols, having up to 5 carbon atoms in their carbon chain skeleton and one hydroxyl group, such as ethanol, propanol, isopropanol, butaneol, iso-butaneol, t-butaneol and pentanol, are considered less desirable solvents or polar solvents due to their skin-irritating effect. Thus, the composition is substantially alcohol-free and includes less than about 5% final concentration of lower alcohols, preferably less than about 2%, more preferably less than about 1%.

### Substantially Non Aqueous

In certain cases, the active agent degrades in the presence of water, and therefore, in such cases the present of water in the composition is not desirable. Thus, in certain preferred embodiments, the composition is substantially non-aqueous. The term “substantially non-aqueous” or “substantially waterless” is intended to indicate that the composition has a water content below about 5%, preferably below about 2%, such as below about 1.5%. In certain other preferred embodiments the composition is non aqueous or waterless.

By non aqueous or waterless is meant that the composition contains no or substantially no, free or unassociated or absorbed water. It will be understood by a person of the art that the waterless solvents and substances miscible with them can be hydrophilic and can contain water in an associated or unfree or absorbed form and may absorb water from the atmosphere and the ability to do so is its hygroscopic water capacity. It is intended that essentially non-aqueous formulations are included within its scope such that the formulations may have present a small amount of water. In some embodiments the composition ingredients are pretreated to reduce, remove or eliminate any residual or associated or absorbed water.

### Shakability

‘Shakability’ means that the composition contains some or sufficient flow to allow the composition to be mixed or remixed on shaking. That is, it has fluid or semi fluid properties. In some very limited cases possibly aided by the presence of silicone it may exceptionally be possible to have a foamable composition which is flowable but not apparently shakable.

### Breakability

A breakable foam is one that is thermally stable, yet breaks under sheer force.

The breakable foam is not “quick breaking”, i.e., it does not readily collapse upon exposure to body temperature environment. Sheer-force breakability of the foam is clearly advantageous over thermally induced breakability, since it allows comfortable application and well directed administration to the target area.

### Humectant

A humectent is a substance that helps retain moisture and also prevents rapid evaporation. Non limiting examples are propylene glycol, propylene glycol derivatives, glycerin, hydrogenated starch hydrosylate, hydrogenated lanolin, lanolin wax, D manitol, sorbitol, sodium 2-pyrrolidone-5-carboxylate, sodium lactate, sodium PCA, soluble collagen, dibutyl phthalate, and gelatin. Other examples may be found in the Handbook of Pharmaceutical Additives published by Gower.

### Moisturizers

A moisturizer, is a substance that helps retain moisture or add back moisture to the skin. Examples are allantoin, petrolatum, urea, lactic acid, sodium PCV, glycerin, shea butter, caprylic/capric/stearic triglyceride, candelilla wax, propylene glycol, lanolin, hydrogenated oils, squalene, sodium hyaluronate and lysine PCA. Other examples may be found in the Handbook of Pharmaceutical Additives published by Gower.

Pharmaceutical compositions may in one or more embodiments usefully comprise in addition a humectant or a moisturizer or combinations thereof.

### Polar Solvent

Optionally, the foamable vehicle further includes at least one polar solvent.

A “polar solvent” is an organic solvent, typically soluble in both water and oil. Certain polar solvents, for example propylene glycol and glycerin, possess the beneficial property of a humectant.

In one or more embodiments, the polar solvent is a humectant.

In one or more embodiments, the polar solvent is a polyol. Polyols are organic substances that contain at least two hydroxy groups in their molecular structure.

In one or more embodiments, the polar solvent contains an diol (a compound that contains two hydroxy groups in its molecular structure), such as propylene glycol (e.g., 1,2-propylene glycol and 1,3-propylene glycol), butaneediol (e.g., 1,4-butaneediol), butaneediol (e.g., 1,3-butaneediol and 1,4-butenediol), butynediol, pentanediol (e.g., 1,5-pentanediol), hexanediol (e.g., 1,6-hexanediol), octanediol (e.g., 1,8-octanediol), neopentyl glycol, 2-methyl-1,3-propanediol, diethylene glycol, triethylene glycol, tetraethylene glycol, dipropylene glycol and dibutylene glycol.

In one or more embodiments, the polar solvent contains a triol (a compound that contains three hydroxy groups in its molecular structure), such as glycerin and 1,2,6-Hexanetriol.

Other non-limiting examples of polar solvents include pyrrolidones, (such as N-methyl-2-pyrrolidone and 1-methyl-2-pyrrolidinone), dimethyl isosorbide, 1,2,6-hexapetriol, dimethyl sulfoxide (DMSO), ethyl proxitol, dimethylacetamide (DMAc) and alpha hydroxy acids, such as lactic acid and glycolic acid.

According to still other embodiments, the polar solvent is a polyethylene glycol (PEG) or PEG derivative that is liquid at ambient temperature, including PEG200 (MW (molecular weight) about 190-210 kD), PEG300 (MW about 285-315 kD), PEG400 (MW about 380-420 kD), PEG600 (MW about 570-630 kD) and higher MW PEGs such as PEG 4000, PEG 6000 and PEG 10000 and mixtures thereof.

Polar solvents are known to enhance the penetration of active agent into the skin and through the skin, and therefore, their inclusion in the composition can be desirable, despite their undesirable skin drying and irritation potential. There is at one level a commonality between the different polar solvents and their penetration enhancement properties. Lower molecular weight alcohols can sometimes be more potent as a solvent, for example by extracting lipids from the skin layers more effectively, which characteristic can adversely affect the skin structure and cause dryness and irritation. Therefore the selection of lower molecular weight alcohols is ideally avoided.

Polar solvents, such as detailed below possess high solubilizing capacity and contribute to the skin penetration of an active agent. Non limiting examples include dimethyl isosorbide polyols, such as glycerol (glycerin), propylene glycol, hexylene glycol, diethylene glycol, propylene glycol n-alkanols, terpenes, di-terpenes, tri-terpenes, limonene, terpene-ol, 1-menthol, dioxolane, ethylene glycol, other glycols, oleyl alcohol, alpha-hydroxy acids, such as lactic acid and glycolic acid, sulfoxides, such as dimethylsulfoxide (DMSO), dimethylformanide, methyl dodecyl sulfoxide, dimethylacetamide, azone (1-dodecylazacycloheptan-2-one), 2-(n-nonyl)-1,3-dioxolane, alkanols, such as dialkylamino acetates, and admixtures thereof. In certain preferred embodiments, the polar solvent is selected from the group consisting of dimethyl isosorbide glycerol (glycerin), propylene glycol, hexylene glycol, terpene-ol, oleyl alcohol, lactic acid and glycolic acid.

### Skin Penetration Enhancer

A “skin penetration enhancer”, also termed herein “penetration enhancer,” is an organic solvent, typically soluble in both water and oil. Examples of penetration enhancer include polyols, such as glycerol (glycerin), propylene glycol, hexylene glycol, diethylene glycol, propylene glycol n-alkanols, terpenes, di-terpenes, tri-terpenes, terpen-ols, limonene, terpene-ol, 1-menthol, dioxolane, ethylene glycol, hexylene glycol, other glycols, sulfoxides, such as dimethylsulfoxide (DMSO), dimethylformanide, methyl dodecyl sulfoxide, dimethylacetamide, dimethylisosorbide, monooleate of ethoxylated glycerides (with 8 to 10 ethylene oxide units), azone (1-dodecylazacycloheptan-2-one), 2-(n-nonyl)-1,3-dioxolane, esters, such as isopropyl myristate/palmitate, ethyl acetate, butyl acetate, methyl proprionate, capric/caprylic triglycerides, octylmyristate, dodecyl-myristate; myristyl alcohol, lauryl alcohol, lauric acid, lauryl lactate ketones; amides, such as acetamide oleates such as triolein; various alkanoic acids such as caprylic acid; lactam compounds, such as azone; alkanols, such as dialkylamino acetates, and admixtures thereof.

According to one or more embodiments, the penetration enhancer is a polyethylene glycol (PEG) or PEG derivative that is liquid at ambient temperature.

### Potent Solvent

In one or more embodiments, the foamable composition includes a potent solvent, in addition to or in place of one of the hydrophobic solvents, polar solvents or emollients of the composition. A potent solvent is a solvent other than mineral oil that solubilizes a specific active agent substantially better than a hydrocarbon solvent such as mineral oil or petrolatum. For example, a potent solvent solubilizes the active agent 5 fold better than a hydrocarbon solvent; or even solubilizes the active agent 10-fold better than a hydrocarbon solvent.

In one or more embodiments, the composition includes at least one active agent in a therapeutically effective concentration; and at least one potent solvent in a sufficient amount to substantially solubilize the at least one active agent in the composition. The term “substantially soluble” means that at least 95% of the active agent has been solubilized, i.e., 5% or less of the active agent is present in a solid state. In one or more embodiments, the concentration of the at least one potent solvent is more than about 40% of the at least one solvent of the composition; or even more than about 60%.

Non-limiting examples of pairs of active agent and potent solvent include: Betamethasone valerate: Practically insoluble in mineral oil (<0.01%); soluble more than 1% in glycofurol; Hydrocortisone butyrate: Practically insoluble in mineral oil (<0.01%); soluble more than 1% in glycofurol; Metronidazole: Practically insoluble in mineral oil (<0.01%); soluble more than 1% in dimethyl isosrbide; Ketoconazole: Practically insoluble in mineral oil (<0.01%); soluble more than 1% in glycofurol, propylene glycol and dimethyl isosrbide; Mupirocin: Practically insoluble in mineral oil (<0.01%); soluble more than 1% in glycofurol, hexylene glycol, dimethyl isosorbide, propylene glycol and polyethylene glycol 400 (PEG 400); Meloxicam, a nonsteroidal anti-inflammatory agent: Practically insoluble in mineral oil (<0.001%); soluble in propylene glycol: 0.3 mg/mL; and in PEG 400:3.7 mg/mL; and Progesterone: Practically insoluble in mineral oil (<0.001%); soluble in PEG 400:15.3 mg/mL.

A non-limiting exemplary list of solvents that can be considered as potent solvents includes polyethylene glycol, propylene glycol, hexylene glycol, butaneediols and isomers thereof, glycerol, benzyl alcohol, DMSO, ethyl oleate, ethyl caprylate, diisopropyl adipate, dimethylacetamide, N-methylpyrrolidone, N-hydroxyethylpyrrolidone, polyvinylpyrrolidone, isosorbide derivatives, such as dimethyl isosorbide, glycofurol and ethoxydiglycol (transcutol) and laurocapram. In one or more embodiments, PPG alkyl ether may act as a potent solvent.

The use of a potent solvent in a foam composition provides an improved method of delivering poorly soluble therapeutic agents to a target area. It is known that low drug solubility results in poor bioavailability, leading to decreased effectiveness of treatment. Foam compositions, for which the solvent includes a potent solvent, increase the levels of the active agent in solution and thus, provide high delivery and improved therapy.

Potent solvents, as defined herein, are usually liquid. Formulations comprising potent solvents and active agents are generally disadvantageous as therapeutics, since their usage involves unwanted dripping and inconvenient method of application; resulting in inadequate dosing. Surprisingly, the foams, which are drip-free, provide a superior vehicle for such active agents, enabling convenient usage and accurate effective dosing.

In one or more embodiments, the present invention the foamable pharmaceutical composition may additionally include a mixture of two or more of the solvents selected from the group of hydrophobic solvents, silicone oils, emollients, polar solvents and potent solvents in an appropriate proportion as would be appreciated to a person skilled in the art.

### Modulating Agent

The term modulating agent is used to describe an agent which can improve the stability of or stabilize a foamable carrier or composition and or an active agent by modulating the effect of a substance or residue present in the carrier or composition.

In one or more embodiments, the modulating agent is used in a water in oil or oil in water emulsion. In one or more other embodiments the modulating agent is used in a unique waterless emulsion.

In certain embodiments, the substance or residue may for example be acidic or basic and potentially alter pH in an emulsion environment or it may be one or more metal ions which may act as a potential catalyst in an emulsion environment.

In certain other embodiments, the substance or residue may for example be acidic or basic and potentially alter an artificial pH in a waterless or substantially non aqueous environment or it may be one or more metal ions which may act as a potential catalyst in a waterless or substantially non aqueous environment.

In one or more embodiments, the modulating agent is used to describe an agent which can affect pH in an aqueous solution. The agent can be any of the known buffering systems used in pharmaceutical or cosmetic formulations as would be appreciated by a man of the art. It can also be an organic acid, a carboxylic acid, a fatty acid an amino acid, an aromatic acid, an alpha or beta hydroxyl acid an organic base or a nitrogen containing compound.

In one or more further embodiments, the modulating agent is used to describe an agent, which is a chelating or sequestering or complexing agent that is sufficiently soluble or functional in the solvent to enable it to “mop up” or “lock” metal ions.

In an embodiment, modulating agent is used to describe an agent which can effect pH in an aqueous solution the term modulating agent more particularly means an acid or base or buffer system or combinations thereof, which is introduced into or is present in and acts to modulate the ionic or polar characteristics and any acidity or basesity balance of an emulsion carrier, composition, foamable carrier or foamable composition or resultant foam.

In other embodiments, modulating agent is used to describe an agent which can effect pH in an aqueous solution the term modulating agent more particularly means an acid or base or buffer system or combinations thereof, which is introduced into or is present in and acts to modulate the ionic or polar characteristics and any acidity or basesity balance of a waterless or substantially non aqueous carrier, composition, foamable carrier or foamable composition or resultant foam.

The substance or residue can be introduced into the formulation from any one or more of the ingredients, some of which themselves may have acidic or basic properties. For example the polymer or solvent may contain basic residues in which case it may be desirable or beneficial to add an acid. Alternatively the surfactant may contain some acid residues in which case the addition of a base may be desirable and beneficial. In some cases more than one ingredient may contain residues which may ameliorate or compound their significance. In some circumstances the active ingredient may favor an acidic pH or more significantly may need to be maintained at a certain acidic pH otherwise it may readily isomerize, chemically react or breakdown, in which case introducing acidic components such as an acidic polymer might be of help. In an embodiment sufficient modulating agent is added to achieve a pH in which the active agent is preferably stable. In another embodiment sufficient modulating agent is added to achieve an artificial pH in which the active agent is preferably stable.

The terms pH, pKa, and pKb, buffers and the like are used in classical measurements of an aqueous solution. Such measurements are artificial in a waterless environment. Nevertheless predictions of artificial pH can be made using dilution techniques of measurements of waterless formulations diluted in water they are formulation sensitive and specific and have to be carefully calibrated with complex formulas.

Waterless medium can be polar and protic yet it does not conform to classical ionic behavior.

In one or more embodiments, the modulating agent comprises an organic compound.

In one or more preferred embodiments, the chelating agent is selected from the group consisting of ethylenediaminetetraacetic acid (EDTA), diethylenetriaminepentaacetic acid (DTPA), hydroxyethylenediaminetriacetic acid (HEDTA), nitrilotriacetic acid (NTA), O,O′-bis(2-aminoethyl)ethyleneglycol-N,N,N′,N′-tetraacetic acid (EGTA), trans-1,2-d iaminocyclohexane-N,N,N′,N′-tetraacetic acid (CyDTA) or a pharmaceutically acceptable salt thereof (normally as a sodium salt), more preferably EDTA, HEDTA and their salts; most preferably EDTA and its salts.

In one or more embodiments, a preferred non limiting example of the chelating agent is EDTA. Typically, the chelating and sequestering agent is present in the composition at a level of up to about 5.0%, preferably 1.0 percent, by weight, of the composition.

In one or more embodiments, the modulating agent may also be a preservative or an antioxidant or an ionization agent. Any preservative, antioxidant or ionization agents suitable for pharmaceutical or cosmetic application may be used. Non limiting examples of antioxidants are tocopherol succinate, propyl galate, butylated hydroxy toluene and butyl hydroxy anisol. Ionization agents may be positive or may be negative depending on the environment and the active agent or composition that is to be protected. Ionization agents may for example act to protect or reduce sensitivity of active agents. Non limiting examples of positive ionization agents are benzyl conium chloride, and cetyl pyridium chloride. Non limiting examples of negative ionization agents are sodium lauryl sulphate, sodium lauryl lactylate and phospholipids.

In one or more embodiments, one or more of the surfactants, polymeric agents, hydrophobic solvents, polar solvents, skin penetration enhancers, potent solvents, emollients humectants, moisturizers, or modulating agents of the composition may affect the color of the composition. In certain embodiments, they may cause enhancement of color or an aspect thereof whilst in other embodiments they may ameliorate the color or an aspect thereof.

### Propellant

The propellant is used to generate foam from the foamable composition. Suitable propellants include volatile hydrocarbons such as butane, propane, isobutane and fluorocarbon gases, or mixtures thereof. In an embodiment, the propellant is a mixture of propane, isobutene and butane. In certain embodiments, fluorohydrocarbon propellants, are useful in the production of a non-flammable foamable composition. Such propellants include, but are not limited to chloro fluoro carbon (CFC) propellants, hydrofluorocarbon (HFC) propellants, such as 1,1,1,2 tetrafluorethane, and 1,1,1,2,3,3,3 heptafluoropropane, 1,1, difluoro ethane and 1,1,1,3,3,3 hexafluoropropane. The propellant makes up about 5-25 wt % of the foamable composition. In some circumstances the propellant may be up to 35%.

In one or more embodiments, foamable compositions comprise a combination of a HFC and a hydrocarbon propellant such as n-butane or mixtures of hydrocarbom propellants such as propane, isobutane and butane.

### Additional Components

In certain embodiments, a composition includes one or more additional components. Such additional components include but are not limited to anti perspirants, anti-static agents, buffering agents, bulking agents, chelating agents, cleansers, colorants, conditioners, deodorants, diluents, dyes, emollients, fragrances, hair conditioners, humectants, occlusive agents, pearlescent aids, perfuming agents, permeation enhancers, pH-adjusting agents, preservatives, protectants, skin penetration enhancers, softeners, solubilizers, sunscreens, sun blocking agents, sunless tanning agents, viscosity modifiers vitamins and flavonoids. As is known to one skilled in the art, in some instances a specific additional component may have more than one activity, function or effect.

In further embodiments the agent is one or more of a colored active agent, a colored excipient, a pigment, a dye, a colorant and a coloring agent.

### Colored Active Agent

In the context herein, active pharmaceutical ingredients and active cosmetic ingredients are collectively termed “active agent” or “active agents.”

In one or more embodiments, the color active agent is the active ingredient. It can be used in the formulation as a suspended solid or in solution, alone or in combination with other active agents. As is known to one skilled in the art, in some instances a specific active agent or color active agent may have more than one activity, function or effect.

Colored active agents can be derived chemically or through extraction from a natural source, such as mineral, plant, or animal sources. The following table provides examples of colored active agents.

| Colored active agent | Color |
|---|---|
| Iodine | Purple/Brown |
| Povidone Iodine | Brown |
| Coal tar extract | Dark brown |
| Hammamelis extract | Dark brown |
| Tetracycline | Bright yellow |
| Minocycline | Yellow |
| Ichthyol (ammonium bituminosulphonate) | Reddish brown |
| Sulfur | Yellow |
| Anthralin | Brown |
| Adriamycin (Doxorubicin) | Red |

Any active agent, which is colored in its raw material state and any active agent which renders noticeable color to a semi-solid formulation upon inclusion in such formulation is suitable for use according to the present invention as a colored active agent.

In one or more embodiments, the colored active agent may be an extract or tincture of one or more beneficial agents that have beneficial properties, for example, when applied to the skin, a body surface, a body cavity or a mucosal surface. The extract can be, for example, alcoholic, hydroalcoholic, propylene glycol, glycerine, dry, press, cold, hot, liquid carbon dioxide, oil or other process known in the art. The extract or tincture may comprise of substances of animal, plant, (such as herb, fruit, vegetable) mineral or other origin. Non-limiting examples are proteins, polypepeptides, sugars, hyularonic acid, and coal tar. Herbal extracts may be from any known therapeutic herb, as listed for example in Herbal Medicines, London: Pharmaceutical Press Electronic Version 2006 or in the American Herbal Association electronic publication Herbal gram or in German Commission E., such as, angelica, calendula, celery, coltsfoot, comfrey, dandelion, jamaica dogwood, kava, marshmallow, prickly ash, northern prickly ash, southern senna, valerian, agrimony, aloe vera, alfalfa, artichoke, avens, bayberry, bloodroot, blue flag, bogbean, boldo, boneset, broom, buchu, burdock, burnet, calamus, calendula, cascara, centaury, cereus, chamomile, german chamomile, roman chamomile, cinnamon, livers, cohosh, black, cohosh, blue, cola, corn silk, couchgrass, cowslip, damiana, devil's claw, drosera, echinacea, elder, elecampane, euphorbia, eyebright, figwort, frangula, fucus, fumitory, garlic, golden seal, gravel root, ground ivy, guaiacum, hawthorn, holy thistle, hops, horehound black, horehound white, horse chestnut hydrangea, ispaghula, juniper, lady's lipper, liferoot lime flower, liquorice, lobelia, mate, meadowsweet, mistletoe, motherwort, myrrh, nettle, parsley, parsley piert, passionflower, pennyroyal, pilewort, plantain, pleurisy root, pokeroot, poplar, pulsatilla, queen's delight, raspberry, red clover, rosemary, sage, sarsaparilla, sassafras, scullcap, senega, shepherd's purse, skunk cabbage, slippery elm, squill, St. john's wort, stone root, tansy, thyme, uva-ursi, vervain, wild carrot, wild lettuce, willow, witch hazel, yarrow and yellow dock.

Without derogating from the generality of classes of colored active agents, a colored active agent may belong to one of the following classes: herbal extracts, mineral extracts, animal extracts, acaricides, age spot and keratose removing agents, allergen, analgesics, local anesthetics, antiacne agents, antiallergic agents, antiaging agents, antibacterials, antibiotics, antiburn agents, anticancer agents, antidandruff agents, antidepressants, antidermatitis agents, antiedemics, antihistamines, antihelminths, antihyperkeratolyte agents, antiinflammatory agents, antiirritants, antimicrobials, antimycotics, antiproliferative agents, antioxidants, anti-wrinkle agents, antipruritics, antipsoriatic agents, antirosacea agents antiseborrheic agents, antiseptic, antiswelling agents, antiviral agents, antiyeast agents, astringents, topical cardiovascular agents, chemotherapeutic agents, corticosteroids, disinfectants, fungicides, hair growth regulators, immunosuppressants, immunoregulating agents, insecticides, insect repellents, keratolytic agents, lactams, metals, metal oxides, mitocides, neuropeptides, non-steroidal anti-inflammatory agents, oxidizing agents, pediculicides, photodynamic therapy agents, retinoids, sanatives, scabicides, self tanning agents, skin whitening agents, asoconstrictors, vasodilators, vitamins, vitamin D derivatives, wound healing agents and wart removers.

### Additional Therapeutic Agent

Several conditions involve a combination of etiological factors, some of which are affected by or require the use of a colored active agent; and other etiological factors that require an additional therapeutic modality. So in one embodiment there is provided at least a color active agent in combination with at least an additional therapeutic agent and in another embodiment there is provided two or more color active agents in combination with or without another therapeutic agent. For example, psoriasis may be treated by a coal tar extract as well as a steroid drug, and therefore combined treatment would be beneficial. Likewise, acne, which involves a microbial infection, excessive keratin production, excessive sebum production and inflammation, can benefit from treatment with a combination of tetracycline, which is yellow or doxorubicin, which is red; and an additional therapeutic agent, selected from the group consisting of an anti-inflammatory agent, a keratolytic agent, a sebostatice agent and a keratolytic agent. Hence, in many cases, the inclusion of an additional therapeutic agent in the composition, contributes to the clinical activity of the colored active agent. Thus, in one or more embodiments, the composition further includes at least one additional therapeutic agent, in a therapeutically effective concentration.

Suitable additional therapeutic agents include but are not limited to active herbal extracts, acaricides, age spot and keratose removing agents, allergen, analgesics, local anesthetics, antiacne agents, antiallergic agents, antiaging agents, antibacterials, antibiotics, antiburn agents, anticancer agents, antidandruff agents, antidepressants, antidermatitis agents, antiedemics, antihistamines, antihelminths, antihyperkeratolyte agents, antiinflammatory agents, antiirritants, antilipemics, antimicrobials, antimycotics, antiproliferative agents, antioxidants, anti-wrinkle agents, antipruritics, antipsoriatic agents, antirosacea agents antiseborrheic agents, antiseptic, antiswelling agents, antiviral agents, antiyeast agents, astringents, topical cardiovascular agents, chemotherapeutic agents, corticosteroids, dicarboxylic acids, disinfectants, fungicides, hair growth regulators, hormones, hydroxy acids, immunosuppressants, immunoregulating agents, insecticides, insect repellents, keratolytic agents, lactams, metals, metal oxides, mitocides, neuropeptides, non-steroidal anti-inflammatory agents, oxidizing agents, pediculicides, photodynamic therapy agents, retinoids, sanatives, scabicides, self tanning agents, skin whitening agents, asoconstrictors, vasodilators, vitamins, vitamin D derivatives, caratenoids, flavenoids, wound healing agents and wart removers. As is known to one skilled in the art, in some instances a specific active agent may have more than one activity, function or effect.

### Colored Excipients, Colorant, Coloring Agents, Pigments, and Dyes

A colorant, or the substance used to give color, is either dye or pigment. Dye, consisting of small molecules, blends with the water-based solution. A water-dye based colorant tints or stains on a molecular level. Because the dye is composed of single molecules it lays flatter on their surface reflecting light more evenly and appearing more vivid.

Pigment consists of larger molecules than that of the dye; therefore the reflection of light received from a pigmented colorant does not appear as vibrant due to the scattering of the reflected light.

Thus, many possible dyes and pigments can be selected for use according to the present invention. Dyes and pigments may be selected, for example, from the list provided in an FDA document, titled “Summary of Color Additives Listed for Use in the United States in Foods, Drugs, Cosmetics, and Medical Devices” which is published in the FDA internet site, http://www.cfsan.fda.gov/˜dms/opa-col2.html. The detailed lists can also be found in Title 21 of the Code of Federal Regulations Parts 73 and 74. Suitable colorants include FD&C colors and D&C colors. Exemplary colorants, listed in the FDA site, include but are not limited to FD&C Blue No. 1 (Dye and Lake), FD&C Blue No. 2 (Dye and Lake), FD&C Green No. 3 (Dye and Lake), FD&C Red No. 3 (Dye), FD&C Red No. 40 (Dye and Lake), FD&C Yellow No. 5 (Dye and Lake), FD&C Yellow No. 6 (Dye and Lake), Orange B, Citrus Red No. 2, Annatto extract, B-Apo-8′-carotenal, Beta-carotene, Beet powder, Bismuth oxychloride, Canthaxanthin, Carmine, Carrot oil, Chromium hydroxide green, Cochineal extract (carmine); Cottonseed flour, toasted partially defatted, cooked; Ferrous gluconate, Ferric ammonium ferrocyanide, Ferric ferrocyanide, Ferrous gluconate, Ferrous lactate, Fruit juice, Grape color extract, β-Apo-8′-carotenal, β-Carotene, Grape skin extract (enocianina), Guanine, Guaiazulene, Henna, Manganese violet, Paprika, Paprika oleoresin, Pyrophillite, Riboflavin, Saffron, Titanium dioxide, Turmeric, Turmeric oleoresin. Other examples of such colorants include, but are not limited to, Red-6 Ca, Red-6 sodium, red iron oxide, Red 21, and Red 27. Preferably, the colorants do not fade upon sun exposure.

Beta carotene is a carotenoid and antioxidant. It is fat soluble and has a strong color. It has a number of therapeutic uses and has been approved for photoprotection and is used for sunburn protection in sensitive individuals.

In another embodiment the carotenoid is a colorless carotenoid, such as phytoene or phytofluene. These colorless carotenoids are found in the skin. It has been suggested that they may play a role in protecting the skin against aging, uv light and oxidative damage. Interestingly their levels are said to be lower in acne, psoriasis and keratosis pilaris. In a further embodiment a combination of colorless and colored carotenoids are employed in a formulation. It is predicted that a foam comprising colorless carotenoid alone without any other color agent would be almost white.

### Colored Modifying Agent

A color modifying agent is an agent which alters one or more of the intensity, luminance, lightness, hue and tone of color of an object/substance or the color effect of a colored active agent, an excipient, a colorant, a coloring agent, a pigment or a dye on or within the object/substance upon or following contact. Any of the known excipients, colorants, coloring agents, pigments or dyes listed above may also act as a color modifying agent for example in relation to modifying the color effect of a colored active agent.

A reactive color modifier is a compound which can react with a certain substance if present in the formulation to form a color. The color modifier is generally about 0.005 to about 20 percent by weight. Useful color modifying compounds include for example, but are not limited to amino acids; substituted ethylenediamines; and mixtures thereof.

### Color Changing Agents and Color Indicators

Color changing agents include agents that change their color and spectroscopic properties in the visible light and/or ultraviolet spectra, or in response to other stimuli. Color changing agents may respond, for example, to moisture or pH, for example, having one color in a moisture-free environment and another color when in an aqueous environment. The color change may be reversible or irreversible. Suitable color changing agents which are moisture and/or pH activated, include for example but are not limited to, D&C Red 21, D&C Red 27.

In one or more embodiments, the color active agent or color changing agent can be an indicator of change in a physical parameter like pH or to determine the extent of a chemical reaction or degradation or be sensitive to light or heat. Such an agent will change color upon sensing a physical or chemical change as are more particularly illustrated below and is referred to as a color or colored indicator. In an embodiment the color indicator may be a diagnostic of a disorder, diagnostic of degradation of the formulation or active agent, diagnostic of loss of protection, or diagnostic of time to remove the formulation and the like.

Indicators of pH are employed in titrations in analytical chemistry and in biological experiments to determine the extent of a chemical reaction. Various pH indicators are known each having their own particular range such that there are indicators available that have a transition range windows that encompass very high pH, very low pH, and many different ranges in between. Color can also be used as an indicator of sterility or lack of it or the presence of an antiseptic. Some active ingredients change color as they react or degrade. Upon exposure to light some indicators change color.

pH indicators are well documented and can be selected for their ability to change color according to a change of pH over a narrow or defined desired range. For example methyl red is red below pH4.4 and yellow above pH 6.2. Examples of other commonly used indicators are gentian violet, methyl yellow, bromophenol blue, congo red, methyl orange, bromocresol green, azollitim, bromocresol purple, bromothymol blue, phenol red, neutral red, naptholphthalein, cresol red, thymol blue, phenolphthalein, thymolphthalein, alizarine yellow, leucomalachite green. Some have more than one transition such as thymol blue. Also multi purpose indicators can be formulated together to cover a wide range of pH.

In an embodiment, the foam is to provide a visual sign to the user. For example, that the user should leave the foam formulation on the target area until and remove it when it changes color. In a further embodiment, the indicator does not stain the skin surface and is readily washed off.

In an embodiment, the foam is to temporary color an area where treatment is to be made. In a certain aspect, the foam upon application temporary colors an area or changes color or becomes non colored to indicate that the area is sterile or otherwise depending upon he indicator used. After a period of time or an event to which the indicator is responsive the color dissipates.

In an embodiment, the foam is for use with a sun screen formulation to indicate whether or not the foam still provides protection. Thus when he foam is no longer effective as a sunscreen the sun sensitive indicator changes color alerting the user to add more.

In an embodiment, the foam is for use with a self tanning formulation to indicate whether or not the foam should be removed.

In an embodiment, the foam forms a protective film and the indicator shows if the foam film is intact or has broken.

In an embodiment, the foam contains an indicator confirming that the formulation is suitable for use. When the product is no longer suitable it changes color for example on breakdown of the active pharmaceutical ingredient.

In an embodiment, the foam contains an indicator which upon application becomes clear. In a further embodiment the indicator changes color in response to temperature. Thus, where body temperature exceeds 39 degrees for example the foam turns a different color say red or where the body temperature fall below 35 it turns another color say green.

In an embodiment, the foam contains an indicator which is photochromic like titanium oxide, which demonstrates photochromic properties in the presence of light from the ultraviolet region to the infrared region. A list of such indicators is seen in U.S. Pat. No. 5,628,934 which is incorporated by reference. In a further embodiment, the foam contains an indicator which is thermochromic

In an embodiment, the foam is non aqueous and contains an indicator that changes color upon exposure to water.

In an embodiment, the foam contains a pH indicator which changes color or becomes colorless on application to the skin. For example a formulation which is slightly alkaline when applied to he skin which is acidic its pH will fall resulting in he color change.

In an embodiment, the foam contains an indicator which is a diagnostic. Any suitable diagnostic may be used to diagnose skin conditions or disorders. In an embodiment the indicator or diagnostic can be indicative of skin penetration.

In an embodiment, the foam contains a color bioactive. An example is astaxanthin, a carotenoid, which is a strong antioxidant that provides a reddish color. It has been asserted that this color protects against ultra-violet light. Another example is anthocyanins, which are water soluble flavonoid pigments and have been said to act as a sunscreen. In addition to being a light attenuator they are said to be powerful antioxidants and are protective from free radicals. Polyphenol antioxidants may be instrumental in combating oxidative stress and can scavenge free radicals. It has been suggested that they have a role in preventing skin aging and in slowing skin wrinkling. Interestingly lower molecular weight antioxidants such as vitamins C, E, ascorbate, and tocopherol as well as lipoic acid are likewise said to exert protective effects against oxidative stress. Tannins are an example of polyphenols, which can be employed medically for example in anti hemorrhoidal compounds.

In one or more embodiments, the color active agent is or is used in combination with a color indicator or diagnostic using any one or more of the types of agents described herein.

### Fields of Applications

The foamable carrier is suitable for treating any inflicted surface. In one or more embodiments, foamable carrier is suitable for administration to the skin, a body surface, a body cavity or mucosal surface, e.g., the cavity and/or the mucosa of the nose, mouth, eye, ear, respiratory system, vagina or rectum (severally and interchangeably termed herein “target site”).

In one embodiment, the disorder is a dermatological disorder, which can be treated by a color active agent.

In another embodiment, the disorder is a dermatological disorder that benefits from the use of a color active agent in conjunction with another active agent, which may also provide a synergistic therapeutic effect.

By selecting a suitable colored active agent, or a combination of at least two colored active agents, or a combination of at lease one colored active agent and at least one additional therapeutic agent, the foamable composition is useful in treating an animal or a human patient having any one of a variety of dermatological disorders, including dermatological pain, dermatological inflammation, acne, acne vulgaris, inflammatory acne, non-inflammatory acne, acne fulminans, nodular papulopustular acne, acne conglobata, dermatitis, bacterial skin infections, fungal skin infections, viral skin infections, parasitic skin infections, skin neoplasia, skin neoplasms, pruritis, cellulitis, acute lymphangitis, lymphadenitis, erysipelas, cutaneous abscesses, necrotizing subcutaneous infections, scalded skin syndrome, folliculitis, furuncles, hidradenitis suppurativa, carbuncles, paronychial infections, rashes, erythrasma, impetigo, eethyma, yeast skin infections, warts, molluscum contagiosum, trauma or injury to the skin, post-operative or post-surgical skin conditions, scabies, pediculosis, creeping eruption, eczemas, psoriasis, pityriasis rosea, lichen planus, pityriasis rubra pilaris, edematous, erythema multiforme, erythema nodosum, grannuloma annulare, epidermal necrolysis, sunburn, photosensitivity, pemphigus, bullous pemphigoid, dermatitis herpetiformis, keratosis pilaris, callouses, corns, ichthyosis, skin ulcers, ischemic necrosis, miliaria, hyperhidrosis, moles, Kaposi's sarcoma, melanoma, malignant melanoma, basal cell carcinoma, squamous cell carcinoma, poison ivy, poison oak, contact dermatitis, atopic dermatitis, rosacea, purpura, moniliasis, candidiasis, baldness, alopecia, Behcet's syndrome, cholesteatoma, Dercum disease, ectodermal dysplasia, gustatory sweating, nail patella syndrome, lupus, hives, hair loss, Hailey-Hailey disease, chemical or thermal skin burns, scleroderma, aging skin, wrinkles, sun spots, necrotizing fasciitis, necrotizing myositis, gangrene, scarring, and vitiligo.

Likewise, the foamable composition is suitable for treating a disorder of a body cavity or mucosal surface, e.g., the mucosa of the nose, mouth, eye, ear, respiratory system, vagina or rectum. Non limiting examples of such conditions include chlamydia infection, gonorrhea infection, hepatitis B, herpes, HIV/AIDS, human papillomavirus (HPV), genital warts, bacterial vaginosis, candidiasis, chancroid, granuloma Inguinale, lymphogranloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, pelvic inflammation, endometritis, salpingitis, oophoritis, genital cancer, cancer of the cervix, cancer of the vulva, cancer of the vagina, vaginal dryness, dyspareunia, anal and rectal disease, anal abscess/fistula, anal cancer, anal fissure, anal warts, Crohn's disease, hemorrhoids, anal itch, pruritus ani, fecal incontinence, constipation, polyps of the colon and rectum.

In an embodiment, the composition is useful for the treatment of an infection. In one or more embodiments, the composition is suitable for the treatment of an infection, selected from the group of a bacterial infection, a fungal infection, a yeast infection, a viral infection and a parasitic infection.

In an embodiment, the composition is useful for the treatment of wound, ulcer and burn.

The composition is also suitable for administering a hormone to the skin or to a mucosal membrane or to a body cavity, in order to deliver the hormone into the tissue of the target organ, in any disorder that responds to treatment with a hormone.

In an embodiment, the disorder is a dermatological disorder, which is common in children. Foam is advantageous in the topical treatment of children, who are sensitive to treatment with a cream or ointment. Color or the absence of color can play a strong part in patient compliance. On the one hand parents may be concerned to use products which do not stain and are white. On the other hand color may support or encourage better compliance in a child patient. By providing a means in or by which a strongly colored formulation is converted into a gentle but attractive shade without undermining or affecting the active ingredients provides an effective solution to the above tension and conflict in and between parent and child perspective. A gentle but attractive color is still attractive to a child whilst its mildness can alleviate the parents concern.

In an embodiment, there is provided a foam composition which unexpectedly reduces the ability of an approximately similar weight of non foam composition having the same or similar amount of color active agent to stain or to cause staining of a garment and further takes longer to stain the garment. In a further embodiment there is provided a stain produced by a foam composition which unexpectedly is easier to clean than a stain derived from an approximately similar weight of non foam composition having the same or similar amount of color active agent to stain or to cause staining of a garment. In a still further embodiment there is provided a less intense stain produced by a foam composition than a stain derived from an approximately similar weight of non foam composition having the same or similar amount of color active agent to stain or to cause staining of a garment. In a still further embodiment there is provided a foam composition that can be readily and quickly wiped off or removed from a garment before a significant stain can be formed when compared to an approximately similar weight of non foam composition having the same or similar amount of color active agent to stain or to cause staining of a garment, which is more quickly absorbed and harder to remove

### Color and Parameters for Color of Intensity, Luminance, Lightness and Hue

One or more of the following parameters or any other internationally recognized parameter and methodology may be used to determine color change. Alternatively and simply color change can be shown visually by comparing two images side by side.

### Color

Color is the perceptual result of light in the visible region of the spectrum, having wavelengths in the region of 400 nm to 700 nm, incident upon the retina. Physical power (or radiance) is expressed in a spectral power distribution (SPD), often in 31 components each representing a 10 nm band.

The human retina has three types of color photoreceptor cone cells, which respond to incident radiation with somewhat different spectral response curves

There are exactly three types of color photoreceptor, so three numerical components are necessary and sufficient to describe a color, providing that appropriate spectral weighting functions are used. This is the concern of the science of colorimetry. In 1931, the Commission Internationale de L'Éclairage (CIE) adopted standard curves for a hypothetical Standard Observer. These curves specify how an SPD can be transformed into a set of three numbers that specifies a color.CIE is a color standard from the Commission Internationale de l'Eclairage based on brightness, hue, and colorfulness.

### Intensity

Intensity is a measure over some interval of the electromagnetic spectrum of the flow of power. Intensity is a linear-light measure. The standard SI unit for luminous intensity is the candela (cd). The candela (cd) is the luminous intensity, in a given direction, of a source that emits monochromatic radiation of a frequency 540·1012 hertz, and has a radiant intensity in that direction of 1/683 watt per steradian.

### Luminance

Brightness is defined by the CIE as the attribute of a visual sensation according to which an area appears to emit more or less light. Because brightness perception is very complex, the CIE defined a more tractable quantity luminance which is radiant power weighted by a spectral sensitivity function that is characteristic of vision.

### What is Lightness

Human vision has a nonlinear perceptual response to brightness. The perceptual response to luminance is called Lightness. It is denoted L* and is defined by the CIE as a modified cube root of luminance.

Stated differently, lightness perception is roughly logarithmic. An observer can detect an intensity difference between two patches when their intensities differ by more than one about percent.

### Hue

According to the CIE hue is the attribute of a visual sensation according to which an area appears to be similar to one of the perceived colors, red, yellow, green and blue, or a combination of two of them. Hue, is what we call “color” in ordinary language, is described on a circular scale. Hue values begin with red at 0 and run through yellow, green, blue, and purple before returning to red at 255.

### Examples of RGB Color Values for “Familiar” Colors.

The color space for computer based applications is often visualised by a unit cube. Each color (red, green, blue) is assigned to one of the three orthogonal coordinate axes in 3D space.

The first column is the descriptive name of the color; the next three columns are the RGB coordinates in the 0 to 255 range as if the components were being stored in one unsigned byte; the last three columns are the RGB color coordinates in the range of 0 to 1 inclusive.

If all three are 0, the resulting color is black; if all three are 255 the resulting color is white.

Whites

| | | | | | | |
|---|---|---|---|---|---|---|
| antique_white | 250 | 235 | 215 | 0.9804 | 0.9216 | 0.8431 |
| azure | 240 | 255 | 255 | 0.9412 | 1.0000 | 1.0000 |
| ivory | 255 | 255 | 240 | 1.0000 | 1.0000 | 0.9412 |
| lavender | 230 | 230 | 250 | 0.9020 | 0.9020 | 0.9804 |

Greys

| | | | | | | |
|---|---|---|---|---|---|---|
| grey | 192 | 192 | 192 | 0.7529 | 0.7529 | 0.7529 |
| light_grey | 211 | 211 | 211 | 0.8275 | 0.8275 | 0.8275 |
| warm_grey | 128 | 128 | 105 | 0.5000 | 0.5000 | 0.4100 |

Blacks

| | | | | | | |
|---|---|---|---|---|---|---|
| black | 0 | 0 | 0 | 0.0000 | 0.0000 | 0.0000 |
| ivory_black | 41 | 36 | 33 | 0.1600 | 0.1400 | 0.1300 |

Reds

| | | | | | | |
|---|---|---|---|---|---|---|
| Alizarin crimson | 227 | 38 | 54 | 0.8900 | 0.1500 | 0.2100 |
| brick | 156 | 102 | 31 | 0.6100 | 0.4000 | 0.1200 |
| English red | 212 | 61 | 26 | 0.8300 | 0.2400 | 0.1000 |
| maroon | 176 | 48 | 96 | 0.6902 | 0.1882 | 0.3765 |
| pink | 255 | 192 | 203 | 1.0000 | 0.7529 | 0.7961 |
| tomato | 255 | 99 | 71 | 1.0000 | 0.3882 | 0.2784 |
| Venetian red | 212 | 26 | 31 | 0.8300 | 0.1000 | 0.1200 |

Browns

| | | | | | | |
|---|---|---|---|---|---|---|
| beige | 163 | 148 | 128 | 0.6400 | 0.5800 | 0.5000 |
| brown | 128 | 42 | 42 | 0.5000 | 0.1647 | 0.1647 |
| chocolate | 210 | 105 | 30 | 0.8235 | 0.4118 | 0.1176 |
| tan | 210 | 180 | 140 | 0.8235 | 0.7059 | 0.5490 |

oranges

| | | | | | | |
|---|---|---|---|---|---|---|
| carrot | 237 | 145 | 33 | 0.9300 | 0.5700 | 0.1300 |
| dark_orange | 255 | 140 | 0 | 1.0000 | 0.5490 | 0.0000 |
| orange | 255 | 128 | 0 | 1.0000 | 0.5000 | 0.0000 |

Yellows

| | | | | | | |
|---|---|---|---|---|---|---|
| banana | 227 | 207 | 87 | 0.8900 | 0.8100 | 0.3400 |
| gold | 255 | 215 | 0 | 1.0000 | 0.8431 | 0.0000 |
| melon | 227 | 168 | 105 | 0.8900 | 0.6600 | 0.4100 |
| yellow | 255 | 255 | 0 | 1.0000 | 1.0000 | 0.0000 |
| yellow_light | 255 | 255 | 224 | 1.0000 | 1.0000 | 0.8784 |

Greens

| | | | | | | |
|---|---|---|---|---|---|---|
| emerald_green | 0 | 201 | 87 | 0.0000 | 0.7900 | 0.3400 |
| forest_green | 34 | 139 | 34 | 0.1333 | 0.5451 | 0.1333 |
| green | 0 | 255 | 0 | 0.0000 | 1.0000 | 0.0000 |
| green_dark | 0 | 100 | 0 | 0.0000 | 0.3922 | 0.0000 |
| green_pale | 152 | 251 | 152 | 0.5961 | 0.9843 | 0.5961 |
| olive | 59 | 94 | 43 | 0.2300 | 0.3700 | 0.1700 |

Cyans

| | | | | | | |
|---|---|---|---|---|---|---|
| cyan | 0 | 255 | 255 | 0.0000 | 1.0000 | 1.0000 |
| turquoise | 64 | 224 | 208 | 0.2510 | 0.8784 | 0.8157 |
| turquoise_dark | 0 | 206 | 209 | 0.0000 | 0.8078 | 0.8196 |
| turquoise_pale | 175 | 238 | 238 | 0.6863 | 0.9333 | 0.9333 |

Blues

| | | | | | | |
|---|---|---|---|---|---|---|
| blue | 0 | 0 | 255 | 0.0000 | 0.0000 | 1.0000 |
| blue_light | 173 | 216 | 230 | 0.6784 | 0.8471 | 0.9020 |
| navy | 0 | 0 | 128 | 0.0000 | 0.0000 | 0.5020 |
| royal_blue | 65 | 105 | 225 | 0.2549 | 0.4118 | 0.8824 |
| sky_blue | 135 | 206 | 235 | 0.5294 | 0.8078 | 0.9216 |
| turquoise_blue | 0 | 199 | 140 | 0.0000 | 0.7800 | 0.5500 |

Magentas

| | | | | | | |
|---|---|---|---|---|---|---|
| blue_violet | 138 | 43 | 226 | 0.5412 | 0.1686 | 0.8863 |
| orchid | 218 | 112 | 214 | 0.8549 | 0.4392 | 0.8392 |
| purple | 160 | 32 | 240 | 0.6275 | 0.1255 | 0.9412 |
| purple medium | 147 | 112 | 219 | 0.5765 | 0.4392 | 0.8588 |
| violet | 143 | 94 | 153 | 0.5600 | 0.3700 | 0.6000 |

### Microemulsions and Nanoemulsions

Microemulsions and nanoemulsion are translucent (or transparent) dispersions of oil and water. Compared to conventional emulsions, microemulsions and nanoemulsion are more thermodynamically stable, making them a favorable vehicle for pharmaceutical compositions, which have to maintain stability for long periods of time. Microemulsions are used, for example, for controlled release of pharmaceutical agents. In contrast to microemulsions they are in a meta-stable state having very fine oil in water dispersions with diameters of <100 nm with good sensorial and biophysical properties such as improved penetration and hydrating power respectively. They and a method of manufacture are more particularly described in US2006/0233721 which is incorporated herein by way of reference. As will be appreciated by a man of the art the methodology may be adapted according to the type of carrier composition.

In one or more embodiments, the composition comprises microemulsions or nano-emulsions in which the hue and intensity of the color are modified compared to regular emulsions.

### Other Characteristics

The foamable compositions are flowable, thermally stable, do not break immediately upon contact with a surface yet break under sheer force, allowing free application without spillage to a body surface or cavity, spread easily and are absorbed quickly. The Foam quality of he foams exemplified herein can be graded as follows:

Grade E (excellent): very rich and creamy in appearance, does not show any bubble structure or shows a very fine (small) bubble structure; does not rapidly become dull; upon spreading on the skin, the foam retains the creaminess property and does not appear watery.

Grade G (good): rich and creamy in appearance, very small bubble size, “dulls” more rapidly than an excellent foam, retains creaminess upon spreading on the skin, and does not become watery.

Grade FG (fairly good): a moderate amount of creaminess noticeable, bubble structure is noticeable; upon spreading on the skin the product dulls rapidly and becomes somewhat lower in apparent viscosity.

Grade F (fair): very little creaminess noticeable, larger bubble structure than a “fairly good” foam, upon spreading on the skin it becomes thin in appearance and watery.

Grade P (poor): no creaminess noticeable, large bubble structure, and when spread on the skin it becomes very thin and watery in appearance.

Grade VP (very poor): dry foam, large very dull bubbles, difficult to spread on the skin.

Topically administrable foams are typically of quality grade E or G, when released from the aerosol container.

Another property of the foam is specific gravity, as measured upon release from the aerosol can. Typically, foams have specific gravity of less than 0.12 g/mL; or less than 0.10 g/mL; or less than 0.08 g/mL, depending on their composition and on the propellant concentration.

Other foamable compositions are described in: U.S. Publication No. 05-0232869, published on Oct. 20, 2005, entitled NONSTEROIDAL IMMUNOMODULATING KIT AND COMPOSITION AND USES THEREOF; U.S. Publication No. 05-0205086, published on Sep. 22, 2005, entitled RETINOID IMMUNOMODULATING KIT AND COMPOSITION AND USES THEREOF; U.S. Publication No. 06-0018937, published on Jan. 26, 2006, entitled STEROID KIT AND FOAMABLE COMPOSITION AND USES THEREOF; U.S. Publication No. 05-0271596, published on Dec. 8, 2005, entitled VASOACTIVE KIT AND COMPOSITION AND USES THEREOF; U.S. Publication No. 06-0269485, published on Nov. 30, 2006, entitled ANTIBIOTIC KIT AND COMPOSITION AND USES THEREOF; U.S. Publication No. 07-0020304, published on Jan. 25, 2007, entitled NON-FLAMMABLE INSECTICIDE COMPOSITION AND USES THEREOF; U.S. Publication No. 06-0193789, published on Aug. 31, 2006, entitled FILM FORMING FOAMABLE COMPOSITION; U.S. patent application Ser. No. 11/732,547, filed on Apr. 4, 2007, entitled ANTI-INFECTION AUGMENTATION OF FOAMABLE COMPOSITIONS AND KIT AND USES THEREOF; U.S. Provisional Patent Application No. 60/789,186, filed on Apr. 4, 2006, KERATOLYTIC ANTIFUNGAL FOAM; U.S. Provisional Patent Application No. 0/815948, filed on Jun. 23, 2006, entitled FOAMABLE COMPOSITIONS COMPRISING A CALCIUM CHANNEL BLOCKER, A CHOLINERGIC AGENT AND A NITRIC OXIDE DONOR; U.S. Provisional Patent Application No. 60/818,634, filed on Jul. 5, 2006, entitled DICARBOXYLIC ACID FOAMABLE VEHICLE AND PHARMACEUTICAL COMPOSITIONS THEREOF; U.S. Provisional Patent Application No. 60/843,140, filed on Sep. 8, 2006, entitled FOAMABLE VEHICLE AND VITAMIN PHARMACEUTICAL COMPOSITIONS THEREOF, all of which are incorporated herein by reference in their entirety. More particularly any of the active ingredients; the solvents; the surfactants; foam adjuvants; penetration enhancers; humectants; moisturizers; and other excipients as well as the propellants listed therein and methodology including preparation of formulations and testing for physical parameters like foam quality, viscosity, hardness, density, collapse time, and creaming or aging etc., can be applied herein and are incorporated by reference.

All % values are provided on a weight (w/w) basis.

The following methodology and examples further exemplify the colored or colorable compositions, foams and colored active agent foamable pharmaceutical carriers, pharmaceutical compositions thereof, methods for preparing the same, and therapeutic uses of the compositions. The examples are for the purposes of illustration only and are not intended to be limiting of the invention. Many variations are contemplated and may be carried out by one of ordinary skill in the art.

### Methodology

A general procedure for preparing foamable compositions is set out in WO 2004/037225, which is incorporated herein by reference.

Emulsion Foam
1. Mix oily phase ingredients and heat to 75° C. to melt all ingredients and obtain homogeneous mixture.
2. Mix polymers in water with heating or cooling as appropriate for specific polymer.
3. Add all other water soluble ingredients to water-polymer solution and heat to 75° C.
4. Add slowly internal phase to external phase at 75° C. under vigorous mixing and homogenize to obtain fine emulsion. Alternatively the external phase is added slowly to the internal phase.
5. Cool to below 40° C. and add sensitive ingredients with mild mixing.
6. Cool to room temperature.

Waterless Foam
1. Dissolve the polymers in the main solvent with heating or cooling as appropriate for specific polymer. Add the all other ingredients and heat to 75° C. to melt and dissolve the various ingredients.
2. Cool to below 40° C. and add sensitive ingredients with mild mixing.
3. Cool to room temperature.

Oily Waterless Foam
1. Mix all ingredients excluding polymers and heat to 75° C. to melt and dissolve and obtain homogeneous mixture.
2. Mix well and cool to below 40° C. and add the polymers and sensitive ingredients with moderate mixing.
3. Cool to room temperature.

Oily Foam with Phospholipids and/or Water
1. Swell the phospholipids in the main oily solvent under mixing for at least 20 minutes until uniform suspension is obtained.
2. Add all other ingredients excluding polymers and heat to 75° C. to melt and dissolve and obtain homogeneous mixture.
3. Mix well and cool to below 40° C. and add the polymers and sensitive ingredients with moderate mixing.
4. Cool to room temperature.
5. In case of polymers dissolved in water or organic solvent, dissolve the polymers in the solvent with heating or cooling as appropriate for specific polymer and add to the oily mixture under vigorous mixing at ˜40° C.

Canisters Filling and Crimping

Each aerosol canister is filled with PFF and crimped with valve using vacuum crimping machine.

Pressurizing

Propellant Filling

Pressurizing is carried out using a hydrocarbon gas or gas mixture.

Canisters are filled and then warmed for 30 sec in a warm bath at 50° C. and well shaken immediately thereafter.

Closure Integrity Test.

Each pressurized canister is subjected to bubble and crimping integrity testing by immersing the canister in a 60° C. water bath for 2 minutes. Canisters are observed for leakage as determined by the generation of bubbles. Canisters releasing bubbles are rejected.

### Tests

By way of non limiting example the objectives of hardness, collapse time and FTC stability tests are briefly set out below as would be appreciated by a person of the art.

### Hardness

LFRA100 instrument is used to characterize hardness. A probe is inserted into the test material. The resistance of the material to compression is measured by a calibrated load cell and reported in units of grams on the texture analyzer instrument display. Preferably at least three repeat tests are made. The textural characteristics of a dispensed foam can effect the degree of dermal penetration, efficacy, spreadability and acceptability to the user. The results can also be looked at as an indicator of softness. Note: the foam sample is dispensed into an aluminum sample holder and filled to the top of the holder.

### Collapse Time

Collapse time (CT) is examined by dispensing a given quantity of foam and photographing sequentially its appearance with time during incubation at 36° C. It is useful for evaluating foam products, which maintain structural stability at skin temperature for at least 1 min.

### Viscosity

Viscosity is measured with Brookfield LVDV-II+PRO with spindle SC4-25 at ambient temperature and 10, 5 and 1 RPM. Viscosity is usually measured at 10 RPM. However, at about the apparent upper limit for the spindle of ˜>50,000CP, the viscosity at 1 RPM may be measured, although the figures are of a higher magnitude.

### FTC (Freeze Thaw Cycles)

To check the foam appearance under extreme conditions of repeated cycles of cooling, heating, (first cycle) cooling, heating (second cycle) etc., commencing with −100° C. (24 hours) followed by +400° C. (24 hours) measuring the appearance and again repeating the cycle for up to three times.

Creaming by Centrifugation:

### 1. Principle of Test

The centrifugation used in this procedure serves as a stress condition simulating the aging of the liquid dispersion under investigation. Under these conditions, the centrifugal force applied facilitates the coalescence of dispersed globules or sedimentation of dispersed solids, resulting in loss of the desired properties of the formulated dispersion.

### 2. Procedure

2.1. Following preparation of the experimental formulation/s, allow to stand at room temperature for ≧24 h.
2.2. Handle pentane in the chemical hood. Add to each experimental formulation in a 20-mL glass vial a quantity of pentane equivalent to the specified quantity of propellant for that formulation, mix and allow formulation to stand for at least 1 h and not more than 24 h.
2.3. Transfer each mixture to 1.5 mL microtubes. Tap each microtube on the table surface to remove entrapped air bubbles.
2.4. Place visually balanced microtubes in the centrifuge rotor and operate the centrifuge at 3,000 rpm for 10 min or at 1,000 rpm for 10 min.

Intra-Canister Uniformity
1. Representative product containers are collected, sample test solutions are prepared and the content of the analyte is determined according to standard methods in the art. Variability of content is characterized as percent difference or relative standard deviation, as appropriate, according to the number of samples evaluated.
2. The results ascertain variability or uniformity within a given container in content of analytes (primarily active pharmaceutical ingredients, but also preservatives) taken from different parts of a pressurized canister drug products
3. Two full canisters were shaken according to product instructions. About 1-3 g of Foam was dispensed from each canister and discarded. Foam sufficient for two replicate sample solution preparations was then dispensed into a glass beaker. This represents the initial sample. A middle portion is then dispensed from each canister being about half the canister contents. This middle dispensed portion may be discarded or collected for testing purposes, as necessary. Foam sufficient for two replicate sample solution preparations was then dispensed into a glass beaker. This represents the final sample. A small amount of formulation remains in the canister. The foam samples were stirred to remove gas/air bubbles. From both the initial and final foam portions from each canister 4 separate sample solutions are prepared and analyzed, 2 from the initial portion and 2 from the final portion. The percent difference is calculated as follows:

$$\frac{\begin{matrix}Difference between content determined in initial \& \\ final portions\end{matrix} }{Mean of content of initial \& final portions} \times 100$$

and the intra canister uniformity evaluated from the results.

### Stock Compositions

Non-limiting examples of how stock solutions are made up with and without API. Other stock solutions may be made using the same methodology by simply varying adding or omitting ingredients as would be appreciated by one of the ordinary skills in the art.

### EXAMPLES

The invention is described with reference to the following examples. This invention is not limited to these examples and experiments. Many variations will suggest themselves and are within the full intended scope of the appended claims. In all the Examples propellant can be added at a concentration of about 3% to about 25%.

### Example 1

### Foamable Water-Free Compositions, Containing Alkanna tinctoria Oil Extract

| Ingredients | % |
|---|---|
| Stearyl Alcohol | 2.00 |
| Hydroxypropyl Cellulose | 2.00 |
| Laureth-4 | 2.00 |
| GMS NE | 2.00 |
| Macrogol Cetostearyl ether | 1.00 |
| PPG-15 stearyl ether | 3.00 |
| Alkanna tinctoria oil extract | 2.44 |
| Propylene glycol | To 100 |

Notes:
The propellant can be added at a concentration of about 3% to about 25%.
The formulations contain polar solvents, which contribute to skin penetration of an active agent

### Example 2

### Decrease of Color Intensity in a Water-Free Foam Containing Alkanna tinctoria Oil Extract

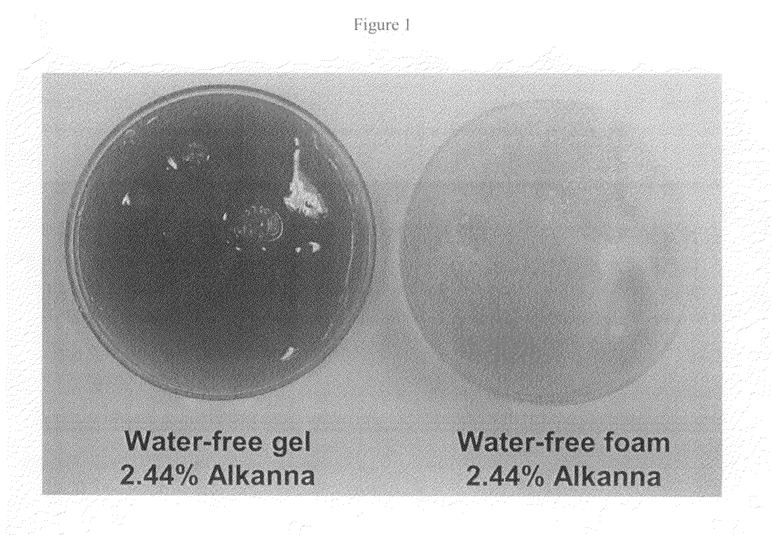

FIG. 1 shows pictures of (1) the composition of Example 1 “as is” (prior to filling into the aerosol container and pressurizing; and (2) the foam produced from the same composition after filling into the aerosol container and pressurizing with 6% hydrocarbon propellant. As shown in the pictures, the color intensity of the foam is significantly lower that the color intensity of the non-foamed composition.

### Example 3

### Foamable Water-Free Compositions, Containing Methylene Blue as Coloring Agent

| Ingredients | % |
|---|---|
| Stearyl Alcohol | 2.00 |
| Hydroxypropyl Cellulose | 2.00 |
| Laureth-4 | 2.00 |
| GMS NE | 2.00 |
| Macrogol Cetostearyl ether | 1.00 |
| PPG-15 stearyl ether | 3.00 |
| Methylene blue | 0.01 |
| Propylene glycol | To 100 |

Notes:
The propellant can be added at a concentration of about 3% to about 25%.
The formulation contains methylene blue, which is a biocompatible coloring agent. It can be used to stain tissues and mark affected areas.

### Example 4

### Decrease of Color Intensity in a Water-Free Foam Containing Alkanna tinctoria Oil Extract

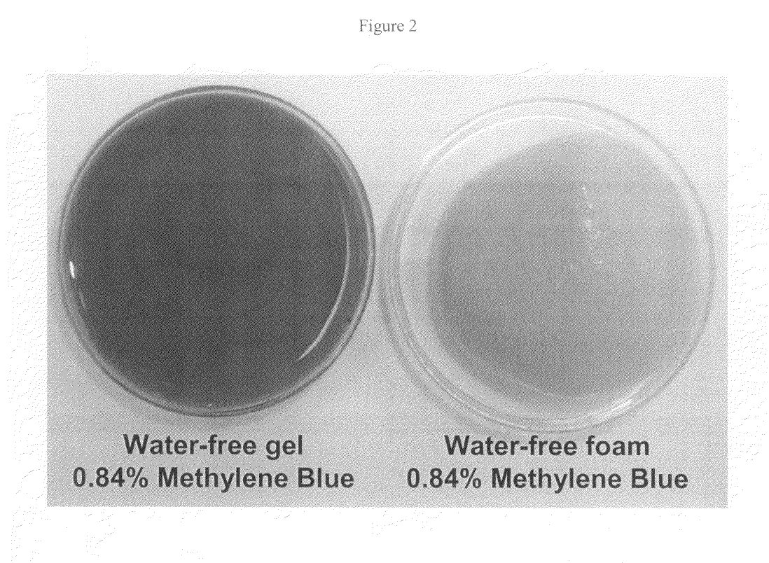

FIG. 2 shows pictures of (1) the composition of Example 3 “as is” (prior to filling into the aerosol container and pressurizing; and (2) the foam produced from the same composition after filling into the aerosol container and pressurizing with 6% hydrocarbon propellant. As shown in the pictures, the color intensity of the foam is significantly lower that the color intensity of the non-foamed composition. However, it is sufficient to mark affected areas.

### Example 5

### Foamable Emulsion Based Compositions, Containing Methylene Blue and Alkanna tinctoria Oil Extract

| Ingredients | % | % |
|---|---|---|
| Mineral oil light | 6.00 | 6.00 |
| Isopropyl myristate | 6.00 | 6.00 |
| Glyceryl monostearate | 0.50 | 0.50 |
| PEG-40 Stearate | 3.00 | 3.00 |
| Stearyl alcohol | 1.00 | 1.00 |
| Hypromellose K100M | 0.30 | 0.30 |
| Xanthan gum | 0.30 | 0.30 |
| Polysorbate 80 | 1.00 | 1.00 |
| Water, purified | 81.30 | 81.30 |
| Preservative | 0.60 | 0.60 |
| Methylene blue | 0.04 | |
| Alkanna tinctoria oil extract | | 2.00 |

Notes:
The propellant can be added at a concentration of about 3% to about 25%.

### Example 6

### Decrease of Color Intensity in a Water-Free Foam Containing Alkanna tinctoria Oil Extract

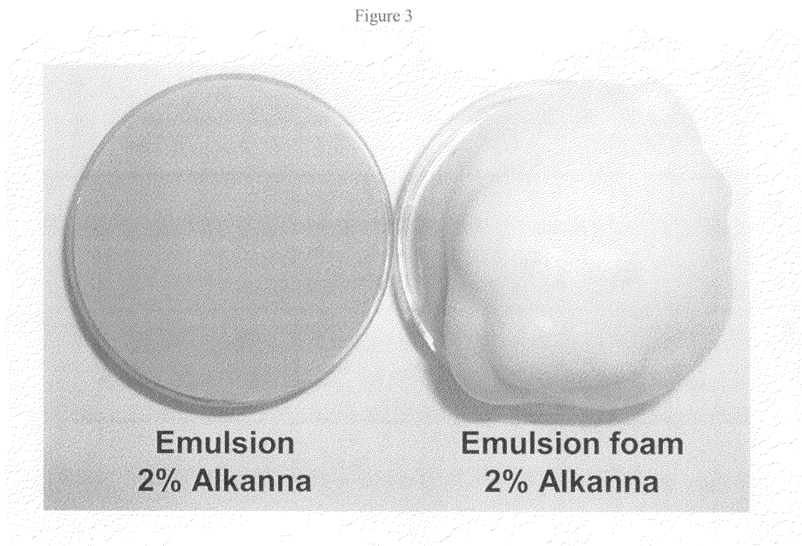

FIG. 3 shows pictures of (1) the composition of Example 5 “as is” (prior to filling into the aerosol container and pressurizing; and (2) the foam produced from the same composition after filling into the aerosol container and pressurizing with 6% hydrocarbon propellant. As shown in the pictures, the color intensity of the foam is significantly lower that the color intensity of the non-foamed composition.

### Example 6A

### Use of a Foam with a Coloring Agent (Methylene Blue) to Mark an Affected Area

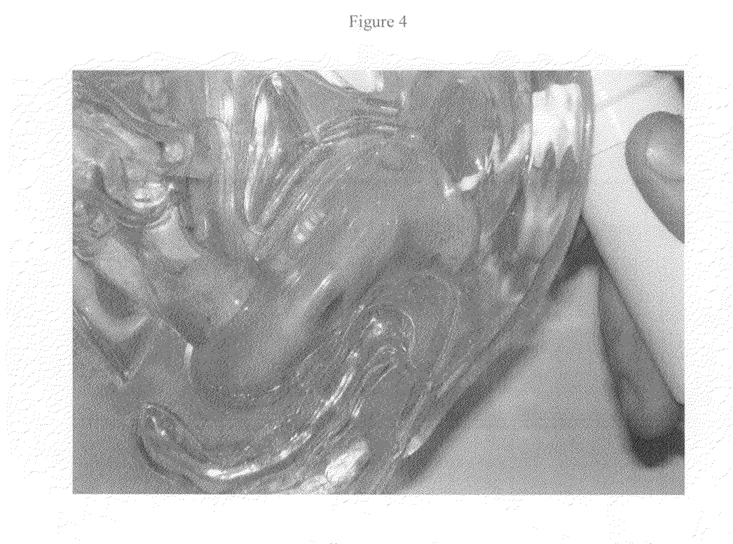

FIG. 4 shows pictures of the foam composition containing Methylene Blue into a model of a vaginal cavity. As shown in the picture, the foam fills the vaginal cavity effectively and markes the area in blue color.

### Example 7

### Foamable Oil in Water Emulsion Vehicle Compositions, Containing Coal Tar Extract

| | CTR001 | CTR002 |
|---|---|---|
| Ingredient name | % W/W | % W/W |
| Coal tar extract (Colored active agent) | 10 | 10 |
| PPG-15 Stearyl ether | — | 3 |
| Isopropyl Myristate | 10 | 5 |
| Octyldodecanol | 12 | 12 |
| Stearyl Alcohol | 2 | 1 |
| Glycerin | — | 3 |
| Lanolin | — | 2 |
| Laureth-4 | — | 2 |
| Emulgin B2 | — | 1.5 |
| Glyceryl Stearate | 1.5 | — |
| PEG-40 Stearate | 3 | — |
| CMC | — | 0.5 |
| Methocel K100M | 0.28 | — |
| Xanthan gum | 0.28 | — |
| Propylene Glycol | — | 5 |
| Polysorbate 60 | 1 | — |
| Water, purified | To 100 | To 100 |

Notes:
The propellant can be added at a concentration of about 3% to about 25%.
The compositions contain a variety of organic carriers, in addition to the PPG alkyl ether.
In the majority of the compositions the surface active agents are solely non-ionic.
The formulations contain polar solvents, which contribute to skin penetration of an active agent

### Example 8

### Foamable Oil in Water Emulsion Compositions, Containing Coal Tar Extract or Anthralin and an Additional Therapeutic Agent

| | CTR002 | CTR004 | CTR005 | CTR006 |
|---|---|---|---|---|
| Ingredient name | % W/W | % W/W | % W/W | % W/W |
| Coal tar extract | 10 | 10 | | |
| (Colored active agent) | | | | |
| Anthralin (Colored | | | 1 | 1 |
| active agent) | | | | |
| Salicylic acid | 5 | 5 | | 5 |
| (Additional therapeutic | | | | |
| agent) | | | | |
| Hydrocortisone | | | 1 | |
| (Additional | | | | |
| therapeutic agent) | | | | |
| PPG-15 Stearyl ether | — | 3 | — | 3 |
| Isopropyl Myristate | 10 | 5 | 10 | 5 |
| Octyldodecanol | 12 | 12 | 12 | 12 |
| Stearyl Alcohol | 2 | 1 | 2 | 1 |
| Glycerin | — | 3 | — | 3 |
| Lanolin | — | 2 | — | 2 |
| Laureth-4 | — | 2 | — | 2 |
| Emulgin B2 | — | 1.5 | — | 1.5 |
| Glyceryl Stearate | 1.5 | — | 1.5 | — |
| PEG-40 Stearate | 3 | — | 3 | — |
| CMC | — | 0.5 | — | 0.5 |
| Methocel K100M | 0.28 | — | 0.28 | — |
| Xanthan gum | 0.28 | — | 0.28 | — |
| Propylene Glycol | — | 5 | — | 5 |
| Polysorbate 60 | 1 | — | 1 | — |
| Water, purified | To 100 | To 100 | To 100 | To 100 |
| Propellant | 8 | 8 | 8 | 8 |

### Example 9

### Decrease of Color Intensity in an Oil-in-Water Emulsion Foam Containing Coal Tar Extract

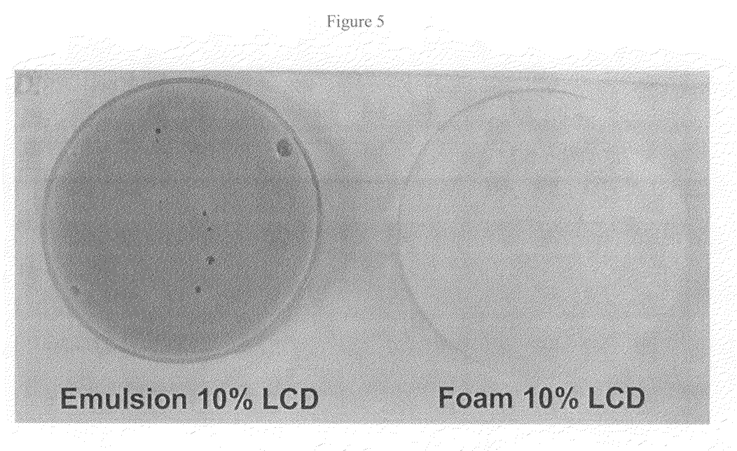

FIG. 5 shows pictures of (1) composition CTR001“as is” (prior to filling into the aerosol container and pressurizing; and (2) the foam produced from the same composition after filling into the aerosol container and pressurizing with 6% hydrocarbon propellant. As shown in the pictures, the color intensity of the foam is significantly lower that the color intensity of the non-foamed composition.

### Example 10

### Decrease of Color Intensity in an Oil-in-Water Emulsion Foam Containing Camellia sinensis Extract

| Ingredient | CCP001 |
|---|---|
| Mineral oil light | 11.00 |
| Polysorbate 80 | 0.90 |
| PEG-40 stearate | 2.60 |
| Xanthan gum | 0.30 |
| Avicel RC 581 | 2.00 |
| Water | 83.10 |
| Camellia sinensis extract | 0.10 |
| Total: | 100.00 |
| Propellant (5515) propane, | 8.00 |
| isobutene and butane mixture | |
| Foam Quality | Excellent |
| Foam Odor | No Odor |
| Foam Shakability | Good |
| Foam Color | Off- |
| | White |
| PFF Color | Mustard |

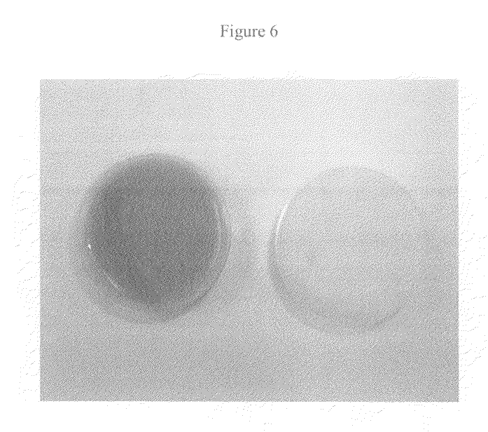

FIG. 6 shows pictures of (1) the composition of Example 10 “as is” (prior to filling into the aerosol container and pressurizing; and (2) the foam produced from the same composition after filling into the aerosol container and pressurizing with 8% hydrocarbon propellant. As shown in the pictures, the color intensity of the foam is significantly lower that the color intensity of the non-foamed composition. As can be seen, the foam is starkly different from the prior to composition.

### Example 11

### Decrease of Color Intensity in Non Aqueous Foams Containing Camellia sinensis Extract

| Ingredient | CCP003 | CCP004 |
|---|---|---|
| Propylene glycol | 97.90 | |
| Peg 400 | | 97.90 |
| Sreareth-2 | 2.00 | 2.00 |
| Camellia sinensis extract | 0.10 | 0.10 |
| Total: | 100.00 | 100.00 |
| Propellant (5515) propane, isobutene | 8.00 | 8.00 |
| and butane mixture | | |
| Foam Quality | Good | Good |
| Foam Odor | No Odor | No Odor |
| Foam Shakability | Good | Good |
| Foam Color | Off-White | Off-White |
| PFF Color | Yellowish | Yellowish |

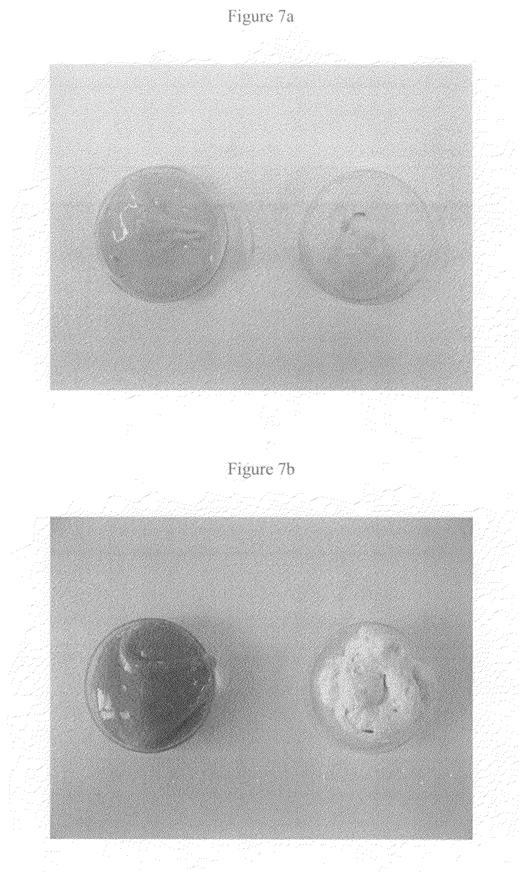

FIGS. 7a and 7b show pictures of (1) the compositions 3 and 4 respectively of Example 11 “as is” (prior to filling into the aerosol container and pressurizing; and (2) the foam produced from the same composition after filling into the aerosol container and pressurizing with 8% hydrocarbon propellant. As shown in the pictures, the color intensity of the foam is significantly lower that the color intensity of the non-foamed composition. In short, the foam is starkly different from the prior to composition.

### Example 12

### Decrease of Color Intensity in Non Aqueous Foams Containing Permethrine Extract

| Ingredients | CCP005 | CCP007C |
|---|---|---|
| Petrolatum (sofmrtic) | | 71.25 |
| Mineral oil light | | 2.85 |
| PPG 15 stearyl ether | | 4.75 |
| Propylene glycol | 93.00 | |
| Cetostearyl alcohol | | 2.85 |
| Span 80 | | 3.80 |
| Behenyl alcohol | | 0.95 |
| Ceteth 20 | | 2.85 |
| GMS | | 0.95 |
| Tween 20 | | 1.90 |
| Sreareth-2 | 2.00 | |
| Aluminum starch octenyl succinate | | 2.85 |
| Permethrin | 5.00 | 5.00 |
| Total: | 100.00 | 100.00 |
| Propellant (5515) propane, | 8.00 | 8.00 |
| isobutene and butane mixture | | |
| Foam Quality | Good | FG |
| Foam Odor | No Odor | No Odor |
| Foam Shakability | Good | Good |
| Foam Color | White | White |
| PFF Color | Off- | Off-White |
| | White | |

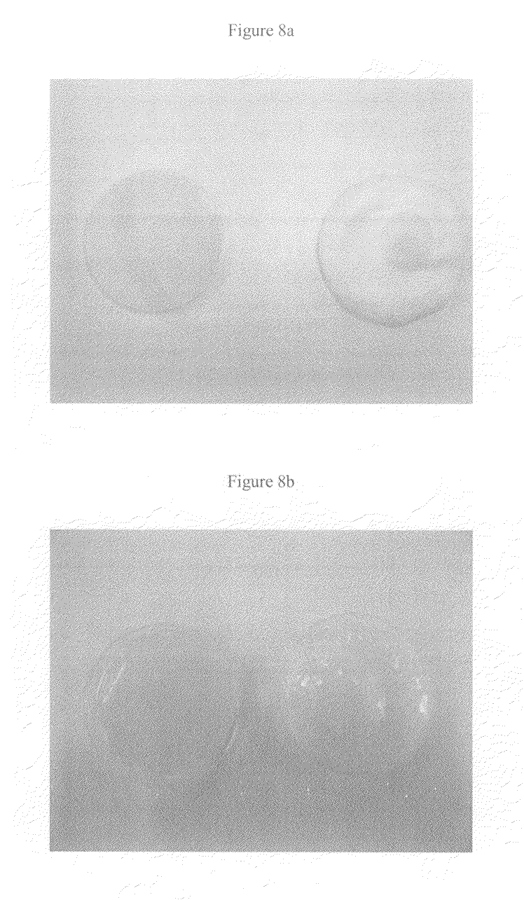

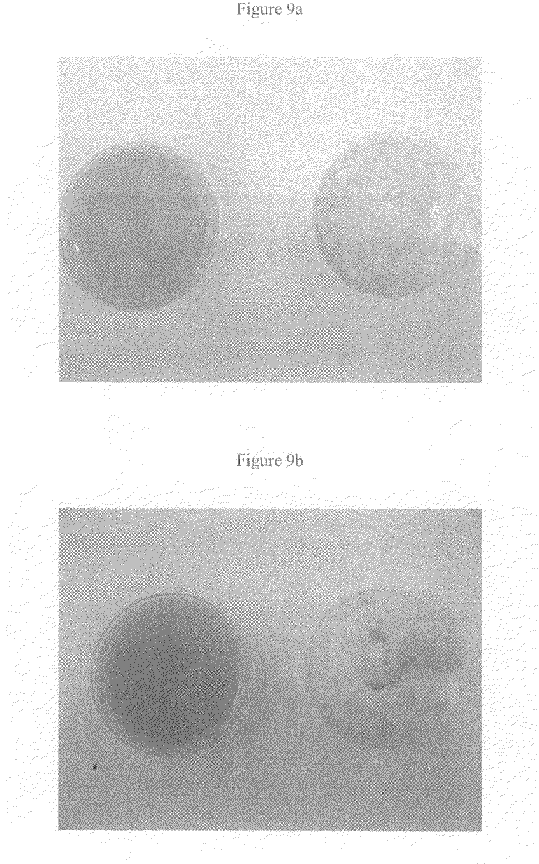

FIGS. 8a and 8b show pictures of (1) the compositions 5 and 7c respectively of Example 12 “as is” (prior to filling into the aerosol container and pressurizing; and (2) the foam produced from the same composition after filling into the aerosol container and pressurizing with 8% hydrocarbon propellant. As shown in the pictures, the color intensity of the foam is only a little or marginally different from that of the color intensity of the non-foamed composition.

### Example 13

### Decrease of Color Intensity in Non Aqueous Foams Containing Minocycline

| Ingredient | CCP006A | CCP007A |
|---|---|---|
| Petrolatum (sofmrtic) | 29.40 | 73.50 |
| Mineral oil light | 38.22 | 2.94 |
| PPG 15 stearyl ether | 14.70 | 4.90 |
| Cetostearyl alcohol | 3.92 | 2.94 |
| Span 80 | | 3.92 |
| Behenyl alcohol | 0.98 | 0.98 |
| Ceteth 20 | 3.92 | 2.94 |
| GMS | 1.96 | 0.98 |
| Tween 20 | | 1.96 |
| Sreareth-2 | 2.94 | |
| Aluminum starch octenyl succinate | 1.96 | 2.94 |
| Minocycline | 2.00 | 2.00 |
| Total: | 100.00 | 100.00 |
| Propellant (5515) propane, isobutene | 8.00 | 8.00 |
| and butane mixture | | |
| Foam Quality | Good | Good |
| Foam Odor | No Odor | No Odor |
| Foam Shakability | Good | Good |
| Foam Color | Yellowish | Yellowish |
| PFF Color | Mutard | Mustard |

FIGS. 9a and 9b show pictures of (1) the compositions 6A and 7A respectively of Example 11 “as is” (prior to filling into the aerosol container and pressurizing; and (2) the foam produced from the same composition after filling into the aerosol container and pressurizing with 8% hydrocarbon propellant. As shown in the pictures, the color intensity of the foam is significantly lower than the color intensity of the non-foamed composition such that the foam is starkly different. As the foam collapses small areas of yellow appear on the surface on the off white foam.

### Example 14

### Decrease of Color Intensity in Non Aqueous Foams Containing Grape Vine Leaf Powder Extract

| Ingredient | CCP002 |
|---|---|
| Mineral oil light | 11.00 |
| Polysorbate 80 | 0.90 |
| PEG-40 stearate | 2.60 |
| Xanthan gum | 0.30 |
| Avicel RC 581 | 2.00 |
| Water | 82.20 |
| Grape Vine Leaf Powder Extract | 1.00 |
| Total: | 100.00 |
| Propellant (5515) propane, isobutene and butane mixture | 8.00 |
| Foam Quality | Excellent |
| Foam Odor | No Odor |
| Foam Shakability | Good |
| Foam Color | Off-White |
| PFF Color | Brown |

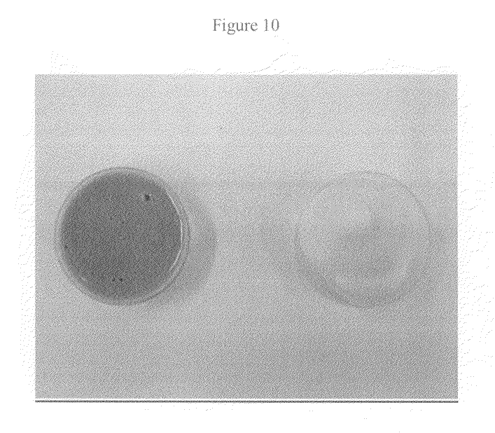

FIG. 10 shows pictures of (1) the composition 2 of Example 14 “as is” (prior to filling into the aerosol container and pressurizing; and (2) the foam produced from the same composition after filling into the aerosol container and pressurizing with 8% hydrocarbon propellant. As shown in the pictures, the color intensity of the foam is significantly lower than the color intensity of the non-foamed composition such that the foam is starkly different.

### Example 15

### Decrease of color Intensity in Non Aqueous Foams Containing Beta Carotene

| Ingredient | CCP008 |
|---|---|
| Mineral oil light | 11.00 |
| Beta carotene | 1.00 |
| PEG-40 stearate | 2.60 |
| Xanthan gum | 0.30 |
| Avicel RC 581 | 2.00 |
| Polysorbate 80 | 0.90 |
| Water pure | 82.20 |
| Total: | 100.00 |
| Propellant (5515) propane, | 8.00 |
| isobutene and butane | |
| mixture | |
| Foam Quality | Excellent |
| Foam Odor | No Odor |
| Foam Shakability | Good |
| Foam Color (T-0) | strong orange |
| PFF Color (T-0) | light orange |
| Foam Color (T-30 min.) | strong orange |

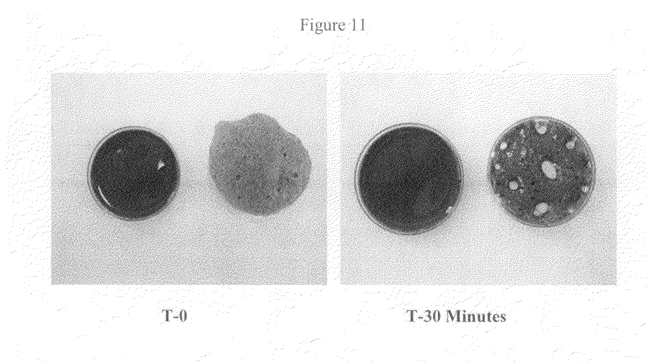

FIG. 11 shows pictures of (1) the composition of Example 15 “as is” (prior to filling into the aerosol container and pressurizing; and (2) the foam produced from the same composition after filling into the aerosol container and pressurizing with 8% hydrocarbon propellant. As shown in the pictures, the color intensity of the foam is significantly lower that the color intensity of the non-foamed composition. However, it is sufficient to mark affected areas. As can be seen, if the foam is left to collapse for 30 minutes the color intensity is all but restored. Although visually the foam intensity is significantly lower when the same weight of non-foamed composition and foam were both placed on a garment and shortly thereafter any excess removed they appeared to have similar marking capacities (not shown).

### Example 16

### Decrease of Color Intensity in Non Aqueous Foams Containing LCD

| Ingredient | CCP006B | CCP007B |
|---|---|---|
| Petrolatum (sofmrtic) | 27.00 | 67.50 |
| Mineral oil light | 35.10 | 2.70 |
| PPG 15 stearyl ether | 13.50 | 4.50 |
| Cetostearyl alcohol | 3.60 | 2.70 |
| Span 80 | | 3.60 |
| Behenyl alcohol | 0.90 | 0.90 |
| Ceteth 20 | 3.60 | 2.70 |
| GMS | 1.80 | 0.90 |
| Tween 20 | | 1.80 |
| Sreareth-2 | 2.70 | |
| Aluminum starch octenyl succinate | 1.80 | 2.70 |
| LCD | 10.00 | 10.00 |
| Total: | 100.00 | 100.00 |
| Propellant (5515) propane, isobutene and | 8.00 | 8.00 |
| butane mixture | | |
| Foam Quality | FG | Poor |
| Foam Odor | Faint Odor | Faint Odor |
| Foam Shakability | Good | Good |
| Foam Color | Yellowish | Yellowish |
| PFF Color | Mustard | Yellow |

In contrast to the results with aqueous LCD seen in Example 9, where the foam quality is better, the difference between the non foam composition and the foam in the non aqueous LCD seen here, where the foam quality is at best fairly good, is small or minimal. It may be, without being bound by any theory, that as the foam quality improves the contrast between the non foam composition and the foam can also increase and vica versa. In other words, as the foam quality increases so the strength or intensity of the foam color appears to decrease.

### Example 17

### Decrease of Color Intensity in an Oil-in-Water Emulsion Foam Containing Quercetin

| Ingredient | AAP030 |
|---|---|
| Mineral oil light | 11.00 |
| Quercetin | 3.00 |
| PEG-40 stearate | 2.60 |
| Xanthan gum | 0.30 |
| Avicel RC 581 | 2.00 |
| Polysorbate 80 | 0.90 |
| Water pure | 80.20 |
| Total: | 100.00 |
| Propellant (5515) | 8.00 |
| propane, isobutene | |
| and butane mixture | |
| Foam Quality | Excellent |
| Foam Odor | No Odor |
| Foam Shakability | Good |
| Foam Color | Yellow |
| PFF Color | Mustard |
| Hardness (g) | 19.42 |
| Viscosity (cp.) | 14688 |
| Density (gr/ml) | 0.039 |
| Collapse time (sec.) | >300 |
| Centrifugation 1K | Creaming |
| | 60% |
| Centrifugation 3K | Creaming |
| | 20% |

As can be seen from the above physical parameters the foam has an overall good set of physical characteristics and shows some resistance to ageing as indicated by centrifugation with no phase separation.

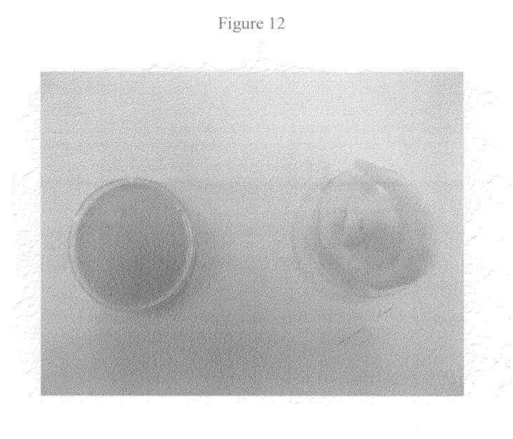

FIG. 12 shows pictures of (1) the composition 30 of Example 17 “as is” (prior to filling into the aerosol container and pressurizing; and (2) the foam produced from the same composition after filling into the aerosol container and pressurizing with 8% hydrocarbon propellant. As shown in the pictures, the color intensity of the foam is significantly lower than the color intensity of the non-foamed composition such that the foam is pleasantly different.

### Example 18

### Decrease of Color Intensity in an Oil-in-Water Emulsion Foam Containing Quercetin and Beta Carotene

| | |
|---|---|
| Chemical name | CCP009 |
| Manufacturing | Feb. 09, 2009 |
| Date | |
| Mineral oil light | 11.00 |
| Beta carotene | 0.50 |
| Quercetin | 1.00 |
| PEG-40 stearate | 2.60 |
| Xanthan gum | 0.30 |
| Avicel RC 581 | 2.00 |
| Polysorbate 80 | 0.90 |
| Water pure | 81.70 |
| Total: | 100.00 |
| Propellant (5515) | 8.00 |
| Foam Quality | Excellent |
| Foam Odor | No Odor |
| Foam Shakability | Good |
| Foam Color | Brown |
| PFF Color | light orange |

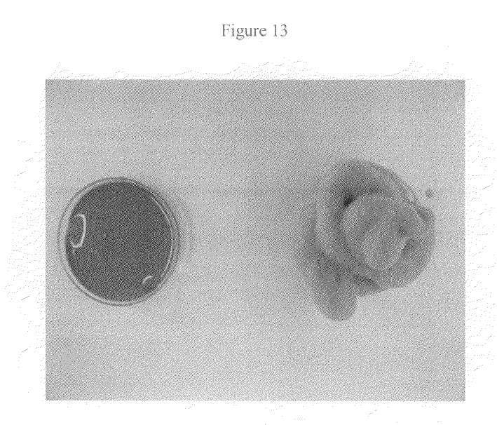

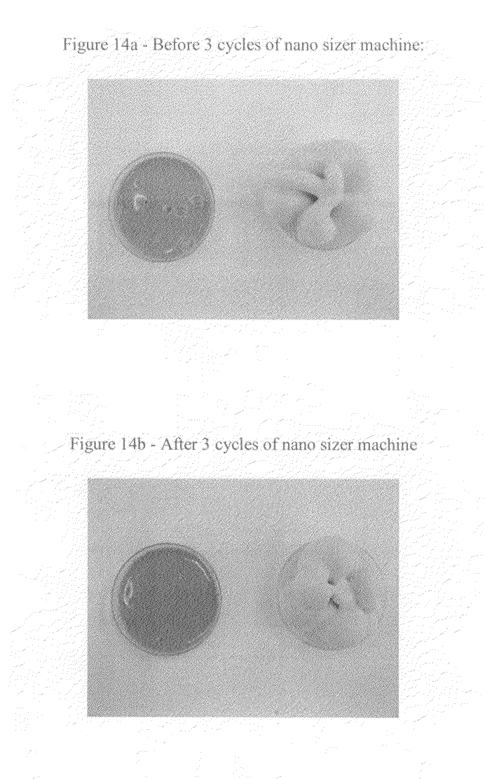

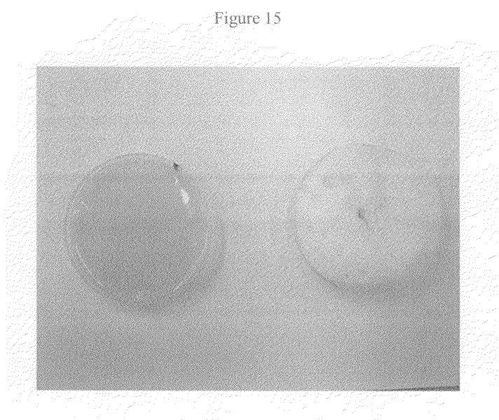

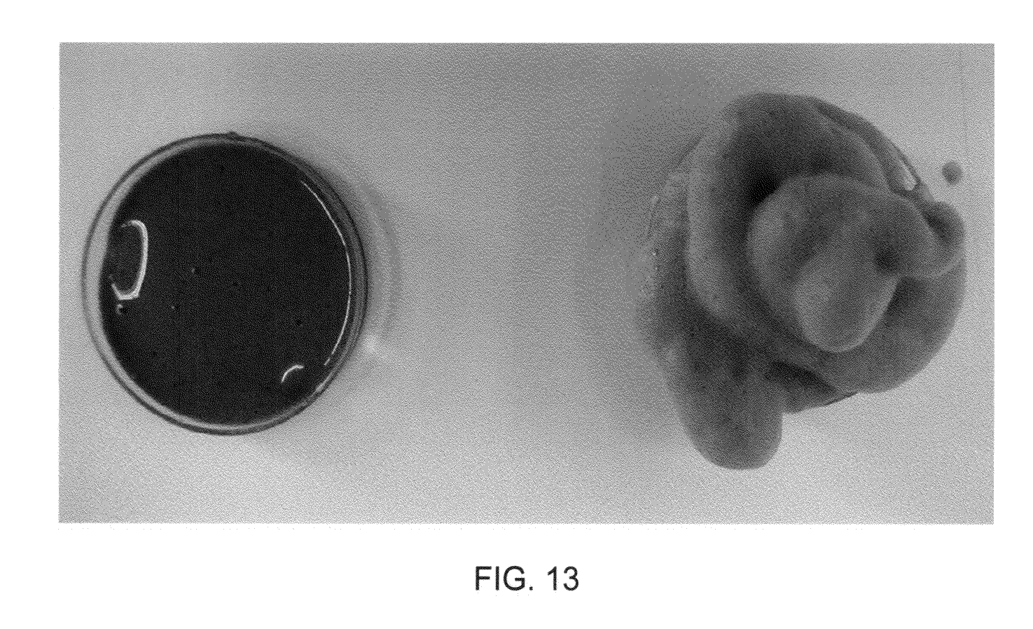

FIG. 13 shows pictures of (1) the composition 9 of Example 18 “as is” (prior to filling into the aerosol container and pressurizing; and (2) the foam produced from the same composition after filling into the aerosol container and pressurizing with 8% hydrocarbon propellant. As shown in the pictures, the color intensity of the foam is significantly lower than the color intensity of the non-foamed composition such that the foam is pleasantly different. Also the drawing highlights the effect on foam appearance when two color active ingredients having different colors are introduced into a foam formulation the resulting foam has a less intense color than its beta carotene parent and a more solid color than its quercitin parent.

### Example 19

### Decrease of Color Intensity in an Oil-in-Water Emulsion Foam and Change in Color Intensity upon Forming a Nano Emulsion Containing Methylene Blue

| | | |
|---|---|---|
| Chemical name | CCP010 | |
| Manufacturing | 04.09.07 | |
| Date | | |
| Mineral oil light | 11.00 | |
| PEG-40 stearate | 2.60 | |
| Xanthan gum | 0.30 | |
| Avicel RC 581 | 2.00 | |
| Polysorbate 80 | 0.90 | |
| Water pure | 82.60 | |
| Sharomix 824 | 0.60 | |
| Total: | 100.00 | |
| Methylene Blue | | |
| Propellant (5515) | 8.00 | |
| Foam Quality | Excellent | Excellent |
| Foam Odor | No Odor | No Odor |
| Foam Shakability | Good | Good |
| Foam Color | blue | blue |
| PFF Color | light blue | light blue* |
| | T-0 | 3 cycles** |

*PFF after 3 cycles-little bluer
**3 cycles of nano sizer machine

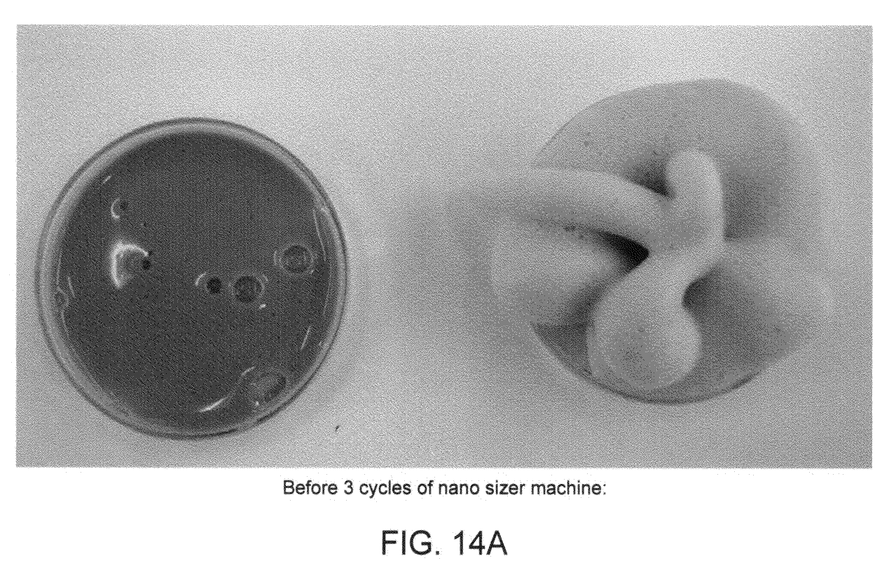

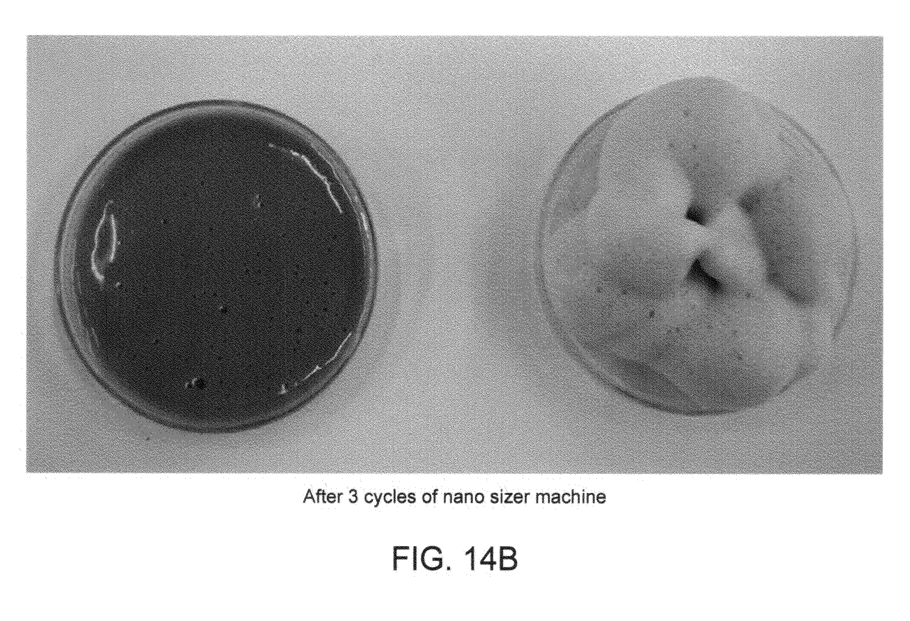

FIGS. 14a and 14b show pictures prior to and after conversion to nano emulsion size of (1) the composition 10 of Example 19 “as is” (prior to filling into the aerosol container and pressurizing; and (2) the foam produced from the same composition after filling into the aerosol container and pressurizing with 8% hydrocarbon propellant. As shown in the pictures, the color intensity of the foam in both cases is significantly lower than the color intensity of the non-foamed composition such that the foam is pleasantly different. Whilst the reduction in emulsion size does not appear to have a significant effect on the foam color surprisingly the non foam nano emulsion composition had a slightly more intense blue color.

### Example 20

### Decrease of Color Intensity in an Oil-in-Water Emulsion Foam Containing Quercetin and Rosmarinic Acid with and Without Ascorbic Acid

| Ingredient | CCP011 | CCP012 |
|---|---|---|
| propylene glycol (PG) | 96.80 | 91.80 |
| steareth 2 | 2.00 | 2.00 |
| Rosmarinic acid | 0.20 | 0.20 |
| Quercetin | 1.00 | 1.00 |
| Ascorbic acid | | 5.00 |
| Total: | 100.00 | 100.00 |
| Propellant (5515) | 8.00 | 8.00 |
| propane butane and | | |
| isobutene mixture | | |
| Foam Quality | Excellent | Excellent |
| Foam Odor | No Odor | No Odor |
| Foam Shakability | Good | Good |
| Foam Color | Yellow | Yellow |
| PFF Color | Off white | Off white |
| Microscope | No | No |
| | Crystals | Crystals |

Note:
All the formulations produced excellent quality foam.
Potentially synergistic combination of quercitin and rosmarinic acid.
A significant excess of two types of reactive antioxidant flavonoids is provided to be available to react in place of vitamin C. It may be the case that if the flavonoids react then the color of the foam and or pff may change thereby providing a self indicator..

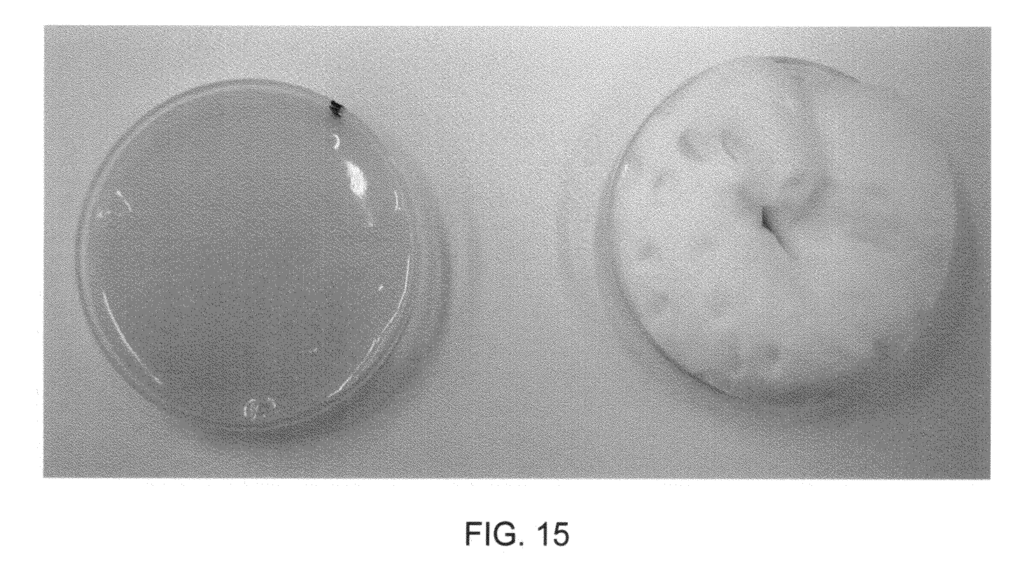

FIG. 15 shows pictures of (1) the composition 12 of Example 20 “as is” (prior to filling into the aerosol container and pressurizing; and (2) the foam produced from the same composition after filling into the aerosol container and pressurizing with 8% hydrocarbon propellant. As shown in the pictures, the color intensity of the foam is significantly lower than the color intensity of the non-foamed composition such that the foam is quite different. Also the drawing highlights the effect on foam appearance when two color active ingredients having different colors are introduced into a foam formulation. The addition of ascorbic acid did not appear to effect the color significantly.

We claim:

1. A colored or colorable topical composition, comprising:
a. a foamable base composition comprising
i. a flowable carrier composition;
ii. a color agent;
1. wherein the color agent is effective to impart, increase, decrease or otherwise affect color of a foam produced from the foamable composition; and
2. wherein the color agent is one or more agents selected from the group consisting of a colored active agent, a colored indicator, a colored excipient, a pigment, a dye, a colorant and a coloring agent.
b. a propellant at a concentration of about 3% to about 25% by weight of the total composition,
wherein the base composition has a first color; and
wherein the foam comprising the colored or colorable topical composition has a second color upon dispensing from an aerosol container, and
wherein the first color and the second color are visually different.

2. The composition of claim 1 wherein the color difference is a difference in one or more of intensity, luminance, lightness and hue.

3. The composition of claim 1 wherein the color difference is about 1% to about 75% of one or more of the internationally recognized parameters for color of intensity, luminance, lightness and hue.

4. The composition of claim 3 wherein the color difference is at least 5%.

5. The composition of claim 3 wherein one or more of the color parameters have decreased.

6. The composition of claim 5 wherein the parameter is selected from the group consisting of intensity and lightness or both.

7. The composition of claim 6 wherein the second color is off white.

8. The composition of claim 1, wherein the flowable carrier composition comprises
at least one carrier, selected from the group consisting of water, an alcohol, a polyol, a polyethylene glycol (PEG), a polar solvent and a hydrophobic carrier comprising an oil, a petrolatum, a silicone oil, a triglyceride and an ester of a fatty acid
at least one stabilizing component, selected from the group consisting of:
a. a surface active agent,
b. a polymeric agent,
optionally a foam adjuvant agent, selected from the group consisting of a fatty alcohol having 15 or more carbons in their carbon chain; a fatty acid having 16 or more carbons in their carbon chain,
wherein the color active agent is sufficiently soluble in the carrier to give expression to the color and the composition is selected from the group consisting of a non aqueous composition, a substantially non aqueous composition or an aqueous composition.

9. The composition of claim 1 further comprising a color modifying agent.

10. The composition of claim 8, wherein the foamable composition comprises an aliphatic alcohol, water, a fatty alcohol and a surface active agent.

11. The composition of claim 8, wherein the foamable composition is an emulsion, comprising water, a hydrophobic carrier, a surface-active agent and a polymeric agent wherein the emulsion is selected from the group consisting of a macro, a micro, and a nano, oil in water or a water in oil emulsion.

12. The composition of claim 11, wherein the hydrophobic carrier is occlusive.

13. The composition of claim 1, wherein the foamable composition is oleaginous.

14. The composition of claim 8, wherein the composition includes more than 50% of a polar solvent

15. The composition of claim 8, wherein the surface active agent is selected from the group consisting of a polysorbate, polyoxyethylene (20) sorbitan monostearate, polyoxyethylene (20) sorbitan monooleate, a polyoxyethylene fatty acid ester, myrj 45, myrj 49, myrj 52 and myrj 59, a polyoxyethylene alkylyl ether, polyoxyethylene cetyl ether, polyoxyethylene palmityl ether, polyethylene oxide hexadecyl ether, polyethylene glycol cetyl ether, brij 38, brij 52, brij 56 brij 72, brij 721 and brij w1, a sucrose ester, a partial ester of sorbitol, sorbitan monolaurate, sorbitan monolaurate a monoglyceride, a diglyceride, isoceteth-20, a sucrose ester, or selected from the group consisting of steareth 2, glyceryl monostearate/peg 100 stearate, glyceryl stearate, steareth-21, peg 40 stearate, polysorbate 60, polysorbate 80, sorbitan stearate, laureth 4, sorbitan monooleate, ceteareth 16 ceteareth 20, steareth 10, steareth 20, ceteth 20, macrogol cetostearyl ether, ceteth 2, peg-30 dipolyhydroxystearate, sucrose distearate, polyoxyethylene (100) stearate, peg 40 stearate, peg 100 stearate, laureth 4, cetomacrogol ether, cetearyl alcohol, cetearyl glucoside, oleyl alcohol, steareth-2, diisopropyl adipate, capric/caprilic triglicerides, polysorbate 20; polysorbate 80, montanov 68 (cetearyl alcohol (and) cetearyl glucoside.), simusol 165 (glyceryl stearate and peg-100 stearate). methyl glucose sequistearate, peg 30 dipolyhydroxystearate, sucrose stearic acid esters, sorbitan laureth, sorbitan stearate, polyglyceryl-10 laurate, epikuuron 80, span 80 and mixtures thereof, and wherein the polymeric agent is selected from the group consisting of locust bean gum, sodium alginate, sodium caseinate, egg albumin, gelatin agar, carrageenin gum, sodium alginate, xanthan gum, quince seed extract, tragacanth gum, guar gum, cationic guars, hydroxypropyl guar gum, starch, an amine-bearing polymer, chitosan, alginic acid, hyaluronic acid, a chemically modified starch, a carboxyvinyl polymer, polyvinylpyrrolidone, polyvinyl alcohol, a polyacrylic acid polymer, a polymethacrylic acid polymer, polyvinyl acetate, a polyvinyl chloride polymer, a polyvinylidene chloride polymer, methylcellulose, hydroxypropyl cellulose, hydroxypropyl methylcellulose, hydroxyethyl cellulose, hydroxy propylmethyl cellulose, methylhydroxyethylcellulose, methylhydroxypropylcellulose, hydroxyethylcarboxymethylcellulose, carboxymethyl cellulose, carboxymethylcellulose carboxymethylhydroxyethylcellulose, a cationic cellulose peg 1000, peg 4000, peg 6000 and peg 8000, Carbopol® 934, Carbopol® 940, Carbopo® 941, Carbopol® 980, Carbopol® 981. hydroxypropylcellulose and carbomer.

16. The composition of claim 1, wherein the colored active agent is selected from a chemically derived active agent and an extract, wherein the extract is from mineral, plant, or animal source.

17. The composition of claim 1, wherein the colored active agents is selected from the group consisting of iodine, povidone Iodine, coal tar extract, hammamelis extract, tetracycline, minocycline, doxorubicin, ichthyol, sulfur, anthralin, camellia sinensis, grape vine leaf powder extract, permethrine, methylene blue, alkanna, beta carotene, rosmarinic acid and quercetin.

18. The composition of claim 1, wherein the colored active agent is an extract from a source selected from angelica, calendula, celery, coltsfoot, comfrey, dandelion, jamaica dogwood, kava, marshmallow, prickly ash, northern prickly ash, southern senna, valerian, agrimony, aloe vera, alfalfa, artichoke, avens, bayberry, bloodroot, blue flag, bogbean, boldo, boneset, broom, buchu, burdock, burnet, calamus, calendula, cascara, centaury, cereus, chamomile, german chamomile, roman chamomile, cinnamon, clivers, cohosh, black, cohosh, blue, cola, corn silk, couchgrass, cowslip, damiana, devil's claw, drosera, echinacea, elder, elecampane, euphorbia, eyebright, figwort, frangula, fucus, fumitory, garlic, golden seal, gravel root, ground ivy, guaiacum, hawthorn, holy thistle, hops, horehound black, horehound white, horse chestnut hydrangea, ispaghula, juniper, lady's lipper, liferoot, lime flower, liquorice, lobelia, mate, meadowsweet, mistletoe, motherwort, myrrh, nettle, parsley, parsley piert, passionflower, pennyroyal, pilewort, plantain, pleurisy root, pokeroot, poplar, pulsatilla, queen's delight, raspberry, red clover, rosemary, sage, sarsaparilla, sassafras, scullcap, senega, shepherd's purse, skunk cabbage, slippery elm, squill, St. john's wort, stone root, tansy, thyme, uva-ursi, vervain, wild carrot, wild lettuce, willow, witch hazel, yarrow and yellow dock.

19. The composition of claim 1, wherein the colored active agent is colored in its raw material state.

20. The composition of claim 1, wherein the colored active agent renders noticeable color to a semi-solid formulation upon inclusion in such formulation.

21. The composition of claim 1, wherein the colored active agent is selected from the group consisting of herbal extracts, mineral extracts, animal extracts, acaricides, age spot and keratose removing agents, allergen, analgesics, local anesthetics, antiacne agents, antiallergic agents, antiaging agents, antibacterials, antibiotics, antiburn agents, anticancer agents, antidandruff agents, antidepressants, antidermatitis agents, antiedemics, antihistamines, antihelminths, antihyperkeratolyte agents, antiinflammatory agents, antiirritants, antimicrobials, antimycotics, antiproliferative agents, antioxidants, anti-wrinkle agents, antipruritics, antipsoriatic agents, antirosacea agents antiseborrheic agents, antiseptic, antiswelling agents, antiviral agents, antiyeast agents, astringents, topical cardiovascular agents, chemotherapeutic agents, corticosteroids, disinfectants, fungicides, hair growth regulators, immunosuppressants, immunoregulating agents, insecticides, insect repellents, keratolytic agents, lactams, metals, metal oxides, mitocides, neuropeptides, non-steroidal anti-inflammatory agents, oxidizing agents, pediculicides, photodynamic therapy agents, retinoids, sanatives, scabicides, self tanning agents, skin whitening agents, asoconstrictors, vasodilators, vitamins, vitamin D derivatives, wound healing agents and wart removers.

22. The composition of claim 1, further comprising an additional component selected from the group consisting of an anti perspirant, an anti-static agent, a buffering agent, a bulking agent, a chelating agent, a colorant, a conditioner, a deodorant, a diluent, a dye, an emollient, fragrance, a humectant, an occlusive agent, a penetration enhancer, a perfuming agent, a permeation enhancer, a pH-adjusting agent, a preservative, a skin penetration enhancer, a sunscreen, a sun blocking agent, a sunless tanning agent, and a vitamins.

23. The composition of claim 1, further comprising an additional therapeutic agent, selected from the group consisting of active herbal extracts, acaricides, age spot and keratose removing agents, allergen, analgesics, local anesthetics, antiacne agents, antiallergic agents, antiaging agents, antibacterials, antibiotics, antiburn agents, anticancer agents, antidandruff agents, antidepressants, antidermatitis agents, antiedemics, antihistamines, antihelminths, antihyperkeratolyte agents, antiinflammatory agents, antiirritants, antilipemics, antimicrobials, antimycotics, antiproliferative agents, antioxidants, anti-wrinkle agents, antipruritics, antipsoriatic agents, antirosacea agents antiseborrheic agents, antiseptic, antiswelling agents, antiviral agents, anti-yeast agents, astringents, topical cardiovascular agents, chemotherapeutic agents, corticosteroids, dicarboxylic acids, disinfectants, fungicides, hair growth regulators, hormones, hydroxy acids, immunosuppressants, immunoregulating agents, insecticides, insect repellents, keratolytic agents, lactams, metals, metal oxides, mitocides, neuropeptides, non-steroidal anti-inflammatory agents, oxidizing agents, pediculicides, photodynamic therapy agents, retinoids, sanatives, scabicides, self tanning agents, skin whitening agents, asoconstrictors, vasodilators, vitamins, vitamin D derivatives, wound healing agents and wart removers.

24. A method of changing the color of a colored or colorable topical composition, comprising:
a. selecting a color agent, a flowable carrier composition a propellant and an aerosol canister,
b. preparing a colored foamable base composition of a first color, comprising:
i. a flowable carrier composition; and
ii. a color agent;
wherein the agent is effective to impart, increase, decrease or otherwise affect color of a foam produced from the foamable composition; and
wherein the agent is one or more agents selected from the group consisting of a colored active agent, a colored excipient, a pigment, a dye, a colorant and a coloring agent,
c. filling the foamable base composition in the aerosol container, closing the container having an aerosol valve, adding a propellant at a concentration of about 3% to about 25% by weight of the total composition, and
d. opening the aerosol valve to release foam of a second color
wherein the first color and the second color are visually different.

25. The method of claim 24 further comprising selecting a color modifying agent and preparing a foamable composition further comprising a color modifying agent.

26. The method of claim 24 further comprising selecting a color indicator, preparing a foamable composition further comprising a color indictor and applying the foam to a target surface wherein the second color will change to a third color upon exposure to a parameter on or in the target surface to which the indicator is responsive and wherein the first color, the second color and the third color are each visually different

27. A method of treating a disorder of a mammalian subject to achieve an improved compliance, comprising:
administering a colored or colorable topical composition to a target site, the colored topical composition, comprising:
a. a foamable base composition comprising
i. a flowable carrier composition;
ii. a color agent
i. wherein the color agent is effective to impart, increase, decrease or otherwise affect color of a foam produced from the foamable composition; and
ii. wherein the color agent is one or more agents selected from the group consisting of a colored active agent, a colored indicator, a colored excipient, a pigment, a dye, a colorant and a coloring agent and;
iii. wherein the color agent comprises at least an effective amount of active agent;
b. a propellant at a concentration of about 3% to about 25% by weight of the total composition,
wherein the base composition has a first color; and
wherein the colored topical composition has a second color after it has been dispensed form an aerosol container, and
wherein the first color and the second color are visually different.

28. The method of claim 27, wherein the target site is selected from the group consisting of the skin, a body cavity, a mucosal surface, the nose, the mouth, the eye, the ear canal, the respiratory system, the vagina and the rectum.

29. The method of claim 27, wherein the disorder is selected from the group consisting of dermatological pain, dermatological inflammation, acne, acne vulgaris, inflammatory acne, non-inflammatory acne, acne fulminans, nodular papulopustular acne, acne conglobata, dermatitis, bacterial skin infections, fungal skin infections, viral skin infections, parasitic skin infections, skin neoplasia, skin neoplasms, pruritis, cellulitis, acute lymphangitis, lymphadenitis, erysipelas, cutaneous abscesses, necrotizing subcutaneous infections, scalded skin syndrome, folliculitis, furuncles, hidradenitis suppurativa, carbuncles, paronychial infections, rashes, erythrasma, impetigo, eethyma, yeast skin infections, warts, molluscum contagiosum, trauma or injury to the skin, post-operative or post-surgical skin conditions, scabies, pediculosis, creeping eruption, eczemas, psoriasis, pityriasis rosea, lichen planus, pityriasis rubra pilaris, edematous, erythema multiforme, erythema nodosum, grannuloma annulare, epidermal necrolysis, sunburn, photosensitivity, pemphigus, bullous pemphigoid, dermatitis herpetiformis, keratosis pilaris, callouses, corns, ichthyosis, skin ulcers, ischemic necrosis, miliaria, hyperhidrosis, moles, Kaposi's sarcoma, melanoma, malignant melanoma, basal cell carcinoma, squamous cell carcinoma, poison ivy, poison oak, contact dermatitis, atopic dermatitis, rosacea, purpura, moniliasis, candidiasis, baldness, alopecia, Behcet's syndrome, cholesteatoma, Dercum disease, ectodermal dysplasia, gustatory sweating, nail patella syndrome, lupus, hives, hair loss, Hailey-Hailey disease, chemical or thermal skin burns, scleroderma, aging skin, wrinkles, sun spots, necrotizing fasciitis, necrotizing myositis, gangrene, scarring, and vitiligo, chlamydia infection, gonorrhea infection, hepatitis B, herpes, HIV/AIDS, human papillomavirus (HPV), genital warts, bacterial vaginosis, candidiasis, chancroid, granuloma Inguinale, lymphogranloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, pelvic inflammation, endometritis, salpingitis, oophoritis, genital cancer, cancer of the cervix, cancer of the vulva, cancer of the vagina, vaginal dryness, dyspareunia, anal and rectal disease, anal abscess/fistula, anal cancer, anal fissure, anal warts, Crohn's disease, hemorrhoids, anal itch, pruritus ani, fecal incontinence, constipation, polyps of the colon and rectum; and wherein the active agent is suitable for treating said disorder.

30. A kit for topical application comprising a colored or colorable topical composition comprising:
a. foamable base composition, comprising:
i. a flowable carrier composition;
ii. a color agent;
1. wherein the color agent is in effective amount to effective to impart, increase, decrease or otherwise affect color of a foam produced from the foamable composition and
2. wherein the color agent is one or more agents selected from the group consisting of a colored active agent, a colored excipient, a pigment, a dye, a colorant and a coloring agent.
b. a propellant at a concentration of about 3% to about 25% by weight of the total composition, and
c. an aerosol canister containing the base composition and propellant
wherein the base composition has a first color; and
wherein the foam comprising the colored topical composition has a second color upon dispensing form an aerosol container, and
wherein the first color and the second color are visually different.

31. Use of a colored or colorable topical composition as a diagnostic, comprising:
a. a foamable base composition comprising
i. a flowable carrier composition;
ii. a color agent;
wherein the color agent is effective to impart, increase, decrease or otherwise affect color of a foam produced from the foamable composition; and
wherein the color agent comprises a color indicator and optionally one or more agents selected from the group consisting of a colored active agent, a colored excipient, a pigment, a dye, a colorant and a coloring agent.
b. a propellant at a concentration of about 3% to about 25% by weight of the total composition,
wherein the base composition has a first color; and
wherein the foam comprising the colored or colorable topical composition has a second color upon dispensing from an aerosol container, and
wherein the first color and the second color are visually different
c. applying the foam to a target surface,
wherein the second color will change to a third color upon exposure to a parameter on or in the target surface to which the indictor is responsive, and wherein the first color, the second color and the third color are each visually different.
